(19) United States
(12) Patent Application Publication
(10) Pub. No.: US 2004/0180845 A1
(43) Pub. Date: Sep. 16, 2004

(54) Methods and compositions for modulating glycogen synthesis and breakdown

(76) Inventors: Christopher B. Newgard, Chapel Hill, NC (US); Ruojing Yang, Durham, NC (US)

Correspondence Address: MYERS BIGEL SIBLEY & SAJOVEC, PO BOX 37428, RALEIGH, NC, 27627, US

(21) Appl. No.: 10/387,767

(22) Filed: Mar. 13, 2003

(51) Int. Cl. A61K 48/00; A61K 39/395

(52) U.S. Cl. 514/44; 424/146.1; 424/94.5

(57) ABSTRACT

The invention provides reagents and methods for reducing hyperglycemia and caloric intake in a subject in need thereof. Also provided are screening methods for identifying compounds that reduce hyperglycemia and/or caloric intake. Further provides are screening methods for identifying compounds that enhance glycogen synthesis without substantially impairing responsiveness to glycogenolytic signals.

### GOVERNMENT SUPPORT

The present invention was made, in part, with the support of grant number P01 DK58398 from the National Institutes of Health. The United States government has certain rights to this invention.

### FIELD OF THE INVENTION

The present invention relates to novel targets and reagents for reducing hyperglycemia and/or caloric intake.

### BACKGROUND OF THE INVENTION

Glycogen targeting subunits of protein phosphatase-1 (PP-1) are scaffolding proteins that organize and regulate key enzymes of glycogen metabolism. Four members of a gene family have been described with varying tissue distributions: the major skeletal muscle isoform, G_{M} (also known as R_{GI} or PPP-1R3), a liver enriched form G_{L} (also known as PPP-1R4), and two forms with wide tissue distribution, protein targeting to glycogen (PTG or PPP-1R5), and PPP-1R6 (Tang et al., (1991) J. Biol. Chem. 266:15782; Doherty (1995) FEBS Lett. 375:294; Printen et al., (1997) Science 275:1475; Doherty et al., (1996) FEBS Lett. 399:339; Newgard et al., (2000) Diabetes 49:1967). All of these proteins share PP-1 and glycogen binding motifs, and to varying degrees, also bind the glycogen metabolizing enzymes glycogen synthase, glycogen phosphorylase, and phosphorylase kinase.

In recent years, the various targeting subunits have been expressed in mammalian cells in culture, or in liver of intact animals, leading to new insights into their relative metabolic potencies and regulatory properties (Newgard et al., (2000) Diabetes 49:1967). This has included work on a novel targeting subunit construct G_{M}ΔC, derived by truncation of the unique 735 amino acid C-terminal domain of native G_{M} (Yang et al., (2002) J. Biol. Chem. 277:1514). It has been found that when expressed in hepatocytes, the targeting subunits stimulate glycogen synthesis in the rank order G_{L}>PTG>G_{M}ΔC>G_{M} (Yang et al., (2002) J. Biol. Chem. 277:1514; Gasa et al., (2000) J. Biol. Chem. 275:26396). Surprisingly, G_{M}ΔC, but not G_{L} or G_{M}, ameliorate glucose intolerance when expressed in liver of rats fed on a high fat diet (Gasa, (2002) J. Biol. Chem. 277:1524). This appears to be explained by the finding that liver cells with overexpressed G_{M}ΔC maintain full responsiveness to glycogenolytic signals such as forskolin and low glucose, unlike cells with overexpressed G_{L} or PTG, which have impaired responsiveness to these agents, resulting in accumulation of large amounts of glycogen in the fasted state (Yang et al., (2002) J. Biol. Chem. 277:1514; Gasa, (2002) J. Biol. Chem. 277:1524; O'Doherty et al., (2000) J. Clin. Invest. 105:479).

There remains a need in the art for methods and reagents for treating hyperglycemia and hyperphagia, in particular, in subjects with diabetes mellitus.

### SUMMARY OF THE INVENTION

The present invention is based, in part, on the finding that glucose homeostasis in diabetic subjects can be achieved by enhancing glycogen storage, even in the presence of low hepatic glucokinase activity. Moreover, glycogen synthesis can be enhanced without abolishing the finely-tuned control mechanisms by which normoglycemia is maintained (e.g., responsiveness to glycogenolytic signals is maintained). Further, the invention is also based on the discovery that enhancement of glycogen storage can reduce caloric intake, in particular in hyperphagic subjects (e.g., subjects with diabetes associated hyperphagia).

The current studies have investigated the properties of G_{M}ΔC by its expression in liver of rats with streptozotocin (STZ)-induced diabetes. Three surprising observations have been made as a result of this work. First, the inventors have found that G_{M}ΔC expression in liver is sufficient to lower blood glucose levels and raise liver glycogen levels to normal in STZ-induced diabetic rats. Second, these corrective effects on hepatic glycogen metabolism and glucose homeostasis occur despite very low levels of liver glucokinase expression in the insulin-deficient animals. Finally, the inventors also find that hepatic G_{M}ΔC expression reduces food intake to normal levels in STZ-diabetic rats, which are otherwise hyperphagic. These findings support the idea that targeting subunits are a viable therapeutic target for lowering of blood glucose levels in diabetes mellitus and for reducing caloric intake.

The normalization of blood glucose and liver glycogen by hepatic G_{M}ΔC expression is remarkable in that it occurs in the face of a 68% reduction in circulating insulin levels. Expression of G_{M}ΔC increases hepatic glucose disposal when the system is challenged by elevated glucose, but allows normal emptying of glycogen stores and protection against hypoglycemia in fasting or other catabolic states. The inventors have also found that overexpression of G_{M}ΔC in normal rats does not result in hypoglycemia following an overnight fast.

Glucokinase is generally thought of as a key regulatory step in hepatic glucose metabolism. Consistent with this idea, patients with maturity-onset diabetes of the young, type 2 (MODY-2) have mutations in their glucokinase gene that affect both insulin secretion and hepatic glucose disposal (Velho et al. (1996) J. Clin. Invest. 98:1755). Furthermore, tissue-specific knock out of the glucokinase gene in liver of mice results in perturbed glucose homeostasis and impaired liver glycogen storage (Postic et al., (1999) J. Biol. Chem. 274:305), while overexpression of the enzyme in liver increases glycogen deposition and lowers blood glucose (O'Doherty et al., (1999) Diabetes 48:2022). It was therefore quite surprising that in the present investigations, G_{M}ΔC expression in liver was able to lower blood glucose levels and normalize liver glycogen content in STZ-injected rats in the face of a dramatic reduction in glucokinase expression. The data clearly establish that normalization of blood glucose can be achieved by manipulation of steps distal to glucokinase, even when this important enzyme is present at very low levels.

The investigations described herein have established that expression of G_{M}ΔC in liver not only reverses glucose intolerance in insulin resistant animals but also normalizes blood glucose in STZ-induced diabetic animals. The surprising demonstration of a glucose lowering effect of G_{M}ΔC in the background of depressed hepatic glucokinase expression places a new focus on drugs that activate liver glycogen storage as a means of controlling blood glucose. That such maneuvers also influence food intake heightens the potential relevance of these findings for treatment of obesity and type 2 diabetes, diseases that are reaching epidemic proportions in much of the world. These investigations demonstrate the feasibility for achieving a therapy for diabetes via improvement of hepatic glucose disposal and stimulation of glycogen storage in a controlled fashion.

Accordingly, as a first aspect; the present invention provides a method of reducing caloric intake by a subject comprising administering to the subject a compound that enhances glycogen synthesis but does not substantially impair responsiveness to glycogenolytic signals, wherein the compound is administered in an amount effective to reduce caloric intake in the subject. In particular embodiments, the subject has diabetes mellitus and/or low hepatic glucokinase activity (e.g., a subject with MODY-2).

As a further aspect, the invention provides a method of identifying a compound that can reduce caloric intake, comprising: contacting a cell with a compound under conditions whereby glycogen synthesis and glycogenolysis can be detected; and detecting whether the compound enhances glycogen synthesis but does not substantially impair responsiveness to glycogenolytic signals, thereby identifying a compound that can reduce caloric intake. In particular embodiments, the cell is a hepatocyte.

The invention also provides whole animal methods of identifying a compound that can reduce caloric intake, comprising: administering a compound to a subject under conditions whereby glycogen synthesis and glycogenolysis can be detected; and detecting whether the compound enhances glycogen synthesis but does not substantially impair responsiveness to glycogenolytic signals, thereby identifying a compound that can reduce caloric intake.

As still a further aspect, the invention provides a method of reducing caloric intake in a subject (e.g., with diabetes mellitus) comprising, administering to the subject an isolated nucleic acid comprising a nucleotide sequence encoding a carboxyl-terminal deleted G_{M} subunit in an amount effective to reduce caloric intake.

As another aspect, the present invention provides a method of reducing hyperglycemia in a subject comprising administering to the subject a compound that enhances glycogen synthesis but does not substantially impair responsiveness to glycogenolytic signals, wherein the compound is administered in an amount effective to reduce hyperglycemia in the subject. In particular embodiments, the subject has diabetes mellitus and/or low hepatic glucokinase activity (e.g., a subject with MODY-2).

As yet a further aspect, the invention provides a method of identifying a compound that can reduce hyperglycemia, comprising: contacting a cell with a compound under conditions whereby glycogen synthesis and glycogenolysis can be detected; and detecting whether the compound enhances glycogen synthesis but does not substantially impair responsiveness to glycogenolytic signals, thereby identifying a compound that can reduce hyperglycemia. In particular embodiments, the cell is a hepatocyte.

The invention also provides whole animal methods of identifying a compound that can reduce hyperglycemia, comprising: administering a compound to a subject under conditions whereby glycogen synthesis and glycogenolysis can be detected; and detecting whether the compound enhances glycogen synthesis but does not substantially impair responsiveness to glycogenolytic signals, thereby identifying a compound that can reduce hyperglycemia.

As still a further aspect, the invention provides a method of reducing hyperglycemia in a subject (e.g., with diabetes mellitus) comprising, administering to the subject an isolated nucleic acid comprising a nucleotide sequence encoding a carboxyl-terminal deleted G_{M} subunit in an amount effective to reduce hyperglycemia.

These and other aspects of the invention are set forth in more detail in the description of the invention below.

### BRIEF DESCRIPTION OF THE DRAWINGS

FIG. 1. Adenovirus-mediated expression of G_{M}ΔC in liver of STZ-injected rats. Animals received a single dose of 60 mg/kg streptozotocin (STZ) or no drug (No STZ). One group of STZ-injected animals was treated with AdCMV-βGAL or no virus (β-GAL/No virus), while a second group was treated with the AdCMV-G_{M}ΔC virus. Panel A. Multiplex PCR analysis of G_{M}ΔC mRNA levels, with α-tubulin as an internal control. Panel B. Immunoprecipitation (anti-FLAG antibody), followed by immunoblot analysis (anti-G_{M} antibody; Tang et al. (1991) J. Biol. Chem. 266:15782-15789) of G_{M}ΔC protein levels. Note that the AdCMV-G_{M}ΔC adenovirus contains a FLAG-tagged version of the G_{M}ΔC cDNA (Yang et al. (2002) J Biol. Chem. 277:1514-1523). Data are shown for 4 animals per group, and are representative of a total of 8 animals in each STZ-treated group and 6 in the No STZ group.

FIG. 2. Lowering of blood glucose levels in STZ-treated animals by hepatic expression of G_{M}ΔC. Panel A. Individual STZ-injected animals prior to (closed diamonds) or 6 days after (open squares) infusion of AdCMV-G_{M}ΔC adenovirus. Panel B. Individual STZ-injected animals prior to (closed diamonds) or after infusion of AdCMV-βGAL virus or no viral infusion (open squares, a mixture of 5 animals that received the AdCMV-βGAL control virus and 3 animals that received no virus). Panel C. Summary of data in panels A and B. The symbol * indicates that blood glucose was lower in animals after treatment with AdCMV-G_{M}ΔC virus than before treatment, with p<0.001.

FIG. 3. Expression of G_{M}ΔC in liver of STZ-injected rats normalizes food intake. The same animals as described in FIG. 2 were used for measurement of food intake. Animals received either a single bolus of 60 mg/kg streptozotocin (STZ-injected) or no streptozotocin injection (No STZ). One group of STZ-injected rats was treated with AdCMV-βGAL adenovirus or received no viral treatment (βGAL/no virus). A separate group of STZ-injected rats received the AdCMV-G_{M}ΔC virus. Food intake was measured during 3 successive 24 h periods, beginning 3 days after viral treatment. Data represent the mean intake/24 h±SEM for 8 animals in each STZ-treated group and 6 in the No STZ group. The symbol * indicates that STZ-injected rats had increased food intake relative to non-injected controls, with p<0.001. The symbol # indicates that AdCMV-G_{M}ΔC-treated, STZ-injected rats had reduced food intake relative to STZ-injected control rats, with p<0.001.

FIG. 4. Circulating leptin levels in STZ-injected rats. The animal treatment groups and labeling are as described in the legend to FIG. 3. Plasma leptin levels were determined by radioimmunoassay 6 days after viral injection. Data represent the mean±SEM for 8 animals in each STZ-injected group and 6 animals in the No STZ group. The symbol * indicates that both the STZ-injected, AdCMV-G_{M}ΔC and STZ-injected control groups had leptin levels lower than the No STZ group, with p<0.02. The symbol # indicates that leptin levels were slightly higher in the STZ-injected, AdCMV-G_{M}ΔC-treated rats than in the STZ-injected control group, with p<0.01.

FIG. 5. Expression of G_{M}ΔC in liver of STZ-injected rats normalizes hepatic glycogen levels. Animals received either a single bolus of 60 mg/kg streptozotocin (STZ-injected) or no streptozotocin injection (No STZ; n=12). One group of STZ-injected rats was treated with AdCMV-βGAL adenovirus or received no viral treatment (AdCMV-βGAL/no virus; n=16). Separate groups of STZ-injected rats received either AdCMV-GMDC (n=8) viruses. Animals were sacrificed for measurement of liver glycogen levels 6 days after viral injection in the ad-lib fed state. The symbol * indicates significant differences relative to STZ-injected controls, with p<0.001. The symbol # indicates significant differences relative to the No STZ, no virus group, with p<0.003.

FIG. 6. Low levels of glucokinase expression in liver of STZ-injected, AdCMV-G_{M}ΔC-treated rats. The animal treatment groups and their labeling are as described in FIG. 3. Animals were sacrificed for measurement of hepatic glucokinase mRNA levels by multiplex RT-PCR. A representative set of samples is shown in Panel A, and quantitative analysis of this data by densitometric scanning is shown in Panel B. The symbol * indicates that STZ-treated animals had lower glucokinase mRNA levels regardless of whether they received AdCMV-G_{M}ΔC or not. Panel C depicts immunoblot analysis of glucokinase protein levels within the same liver samples as used for the RNA analysis in Panels A and B.

FIG. 7. Sequences of G_{M} Targeting Subunit. Nucleotide (SEQ ID NO:7, Panel A) and amino acid (SEQ ID NO:8, Panel B) sequences of rabbit G_{M}-targeting subunit (GenBank Accession M65109; Tang et al., (1991) J. Biol. Chem. 266:15782).

FIG. 7. Sequence of G_{M}ΔC. Nucleotide (SEQ ID NO:9, Panel A) and amino acid (SEQ ID NO:10, Panel B) sequences of a carboxyl terminal deleted G_{M} subunit derived from the sequences of FIG. 7 by truncation of the carboxyl terminal 735 amino acids is shown.

### DETAILED DESCRIPTION OF THE PREFERRED EMBODIMENTS

The present invention will now be described with reference to the accompanying drawings, in which preferred embodiments of the invention are shown. This invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the disclosure, which do not depart from the instant invention.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference in their entirety.

Except as otherwise indicated, standard methods can be used for the production of viral and non-viral vectors, manipulation of nucleic acid sequences, production of transformed cells, and the like according to the present invention. Such techniques are known to those skilled in the art. See, e.g., SAMBROOK et al., MOLECULAR CLONING: A LABORATORY MANUAL 2nd Ed. (Cold Spring Harbor, N.Y., 1989); F. M. AUSUBEL et al. CURRENT PROTOCOLS IN MOLECULAR BIOLOGY (Green Publishing Associates, Inc. and John Wiley & Sons, Inc., New York).

I. Definitions.

As used in the description of the invention and the appended claims, the singular forms “a,” “an” and “the” are intended to include the plural forms as well, unless the context clearly indicates otherwise.

As used herein, the term “caloric intake” indicates the total number of calories ingested, typically in the form of feed or food, by a subject for a specified time period. A “reduction” in caloric intake or “reducing” caloric intake is intended to refer to a decrease in the total number of calories ingested by the subject during a specified time period, e.g., at least about a 5%, 10%, 15%, 20%, 25%, 30%, 35%, 40%, 50% or more diminishment in caloric intake. The reduction in caloric intake can be associated with appetite suppression or induction of satiety. In particular embodiments, the reduction in caloric intake refers to a reduction or normalization in caloric intake by a subject with hyperphagia, e.g., the hyperphagia associated with Type I diabetes or streptozotocin (STZ) administration. In other particular embodiments, the reduction in caloric intake refers to a reduction in caloric intake by a subject that is excreting glucose into its urine.

By “reducing hyperglycemia” it is meant a decrease in the severity and/or the incidence of hyperglycemia, including acute hyperglycemic episodes and chronic hyperglycemic states.

The term “modulate,” “modulates” or “modulation” refers to enhancement (e.g., an increase) or inhibition (e.g., a reduction) in the specified activity.

The term “enhance,” “enhances,” “enhancing” or “enhancement” with respect to glycogen synthesis refers to an increase in glycogen synthesis (e.g., at least about a 1.5-fold, 2-fold, 3-fold, or even four-fold or more increase) as compared with an appropriate control. In particular embodiments, insulin-stimulated and/or glucose-stimulated glycogen synthesis is enhanced. In other particular embodiments, hepatic and/or skeletal muscle glycogen synthesis is enhanced. Glycogen synthesis may be evaluated in cultured cells or in vivo. Likewise, glycogen synthesis may be assessed by any method known in the art, for example, by assessing glycogen stores in the cell or tissue, by measuring the abundance and/or activity of enzymes involved in glycogen synthesis (e.g., glycogen synthase) and the like. Glycogen synthesis can also be indirectly assessed by measuring decreases in blood glucose concentration (or glucose concentration in the cell culture medium).

A “glycogen synthesis enhancer” is a compound that enhances glycogen synthesis in a cell-based, organ/tissue, and/or intact animal system.

A compound that “does not substantially impair responsiveness to glycogenolytic signals” is a compound that does not unduly interfere with one or more normal regulatory mechanisms or signals that trigger glycogen breakdown to maintain blood glucose homeostasis (e.g., in the liver and/or skeletal muscle). By “does not substantially impair” it is meant a reduction or decrease of more than about 20%, 25%, 30%, 40%, 50% or more in glycogen breakdown in response to a specific signal or set of signals (e.g., in response to a particular concentration of glucose, glucagon, insulin, and/or catecholamine in the blood or medium). Glycogenolytic signals include, but are not limited to, hypoglycemia (or low glucose concentrations in culture medium), glucagon, catecholamines, forskolin, low insulin concentrations, intracellular calcium and effectors thereof, and the like. Thus, for example, a compound that does not substantially impair responsiveness to glycogenolytic signals can be a compound that does not substantially impair glycogen breakdown triggered by hypoglycemia. Glycogenolysis can be evaluated in cell-free, in vitro (i.e., in cultured cells), and in vivo systems. Further, glycogenolysis can be evaluated by any method known in the art, for example, by measuring reductions in glycogen stores, by measuring the abundance and/or activity of enzymes involved in glycogenolysis (e.g., glycogen phosphorylase) and the like. Glycogenolysis can also be indirectly assessed by measuring elevations in blood glucose concentration (or glucose concentration in the cell culture medium).

As used herein, the term “diabetes” is used interchangeably with the term “diabetes mellitus.” The terms “diabetes” and “diabetes mellitus” are intended to encompass both insulin dependent and non-insulin dependent (Type I and Type II, respectively) diabetes mellitus as well as other forms of diabetes mellitus such as Maturity Onset Diabetes of the Young, type 2 (MODY-2), unless a particular condition is specifically indicated.

“Low glucokinase activity” may be a result of a low abundance and/or activity of glucokinase (e.g., in the liver) and may further be a result of a genetic defect in the gene encoding the glucokinase gene, e.g., as is found in subjects with MODY-2 (see, e.g., Velho et al., (1996) J. Clin. Invest. 98:1755). Low hepatic glucokinase activity is also typically associated with STZ treatment and untreated Type I diabetes. By “low glucokinase” activity, it is meant an activity level that is less than about 75%, 50%, 25%, or even less of the glucokinase activity in normal liver extracts.

The term “glucose tolerance” refers to a state in which there is proper functioning of the homeostatic mechanisms by which insulin is secreted in response to an elevation in serum glucose concentrations. Impairment in this system results in transient hyperglycemia as the organism is unable to maintain normoglycemia following a glucose load (for example, a carbohydrate containing meal) because of insufficient secretion of insulin from the islet β-cells or because of insensitivity of target tissues to circulating insulin.

“An improvement in glucose tolerance” is a level of improvement that provides some clinical benefit to the subject. Glucose tolerance can be assessed by methods known in the art, such as for example, the oral glucose tolerance test which monitors serum glucose concentrations following an oral glucose challenge. In particular embodiments, an “improvement in glucose tolerance” can result in normalization of fasting or baseline serum glucose concentrations, a reduction in maximal serum glucose concentrations, and/or an improved temporal response to a glucose challenge.

A “transgenic” non-human animal is a non-human animal that comprises a foreign nucleic acid incorporated into the genetic makeup of the animal, such as for example, by stable integration into the genome or by stable maintenance of an episome (e.g., derived from EBV).

A “therapeutically effective” amount as used herein is an amount that provides some improvement or benefit to the subject. Alternatively stated, a “therapeutically-effective” amount is an amount that will provide some alleviation, mitigation, or decrease in at least one clinical symptom in the subject (e.g., reduced insulin resistance, reduced hyperglycemia, or improved glucose tolerance in a diabetic subject; reduced caloric intake in an obese or hyperphagic subject). Those skilled in the art will appreciate that the therapeutic effects need not be complete or curative, as long as some benefit is provided to the subject.

By the terms “treating” or “treatment of,” it is intended that the severity of the patient's condition is reduced or at least partially improved or modified and that some alleviation, mitigation or decrease in at least one clinical symptom is achieved.

As used herein, a “vector” or “delivery vector” can be a viral or non-viral vector that is used to deliver a nucleic acid to a cell, tissue or subject.

A “recombinant” vector or delivery vector refers to a viral or non-viral vector that comprises one or more heterologous nucleotide sequences (i.e., transgenes), e.g., two, three, four, five or more heterologous nucleotide sequences. Generally, the recombinant vectors of the invention encode a carboxyl terminal deleted G_{M} subunit, but can also comprise one or more additional heterologous sequences.

As used herein, the term “viral vector” or “viral delivery vector” can refer to a virus particle that functions as a nucleic acid delivery vehicle, and which comprises the vector genome packaged within a virion. Alternatively, these terms can be used to refer to the vector genome when used as a nucleic acid delivery vehicle in the absence of the virion.

A viral “vector genome” refers to the viral genomic DNA or RNA, in either its naturally occurring or modified form. A “recombinant vector genome” is a viral genome (e.g., vDNA) that comprises one or more heterologous nucleotide sequence(s).

A “heterologous nucleotide sequence” will typically be a sequence that is not naturally-occurring in the vector. Alternatively, a heterologous nucleotide sequence can refer to a sequence that is placed into a non-naturally occurring environment (e.g., by association with a promoter with which it is not naturally associated).

By “infectious,” as used herein, it is meant that a virus can enter a cell by natural transduction mechanisms and express viral genes and/or nucleic acids (including transgenes). Alternatively, an “infectious” virus is one that can enter the cell by other mechanisms and express the genes encoded by the viral genome therein. As one illustrative example, the vector can enter a target cell by expressing a ligand or binding protein for a cell-surface receptor in the virion or by using an antibody(ies) directed against molecules on the cell-surface followed by internalization of the complex.

As used herein, the term “polypeptide” encompasses both peptides and proteins, unless indicated otherwise.

A “fusion polypeptide” is a polypeptide produced when two heterologous nucleotide sequences or fragments thereof coding for two (or more) different polypeptides not found fused together in nature are fused together in the correct translational reading frame. Illustrative fusion polypeptides include fusions of a carboxyl terminal deleted G_{M} subunit to all or a portion of glutathione-S-transferase, maltose-binding protein, or a reporter protein (e.g., Green Fluorescent Protein, β-glucuronidase, β-galactosidase, luciferase, etc.).

As used herein, a “functional” polypeptide is one that retains at least one biological activity normally associated with that polypeptide (e.g., a functional G_{M} targeting subunit retains the ability to bind PP-1, to bind glycogen, and/or to target PP-1 to the glycogen particle). Preferably, a “functional” polypeptide retains all of the activities possessed by the unmodified peptide. By “retains” biological activity, it is meant that the polypeptide has at least about 50%, 60%, 75%, 85%, 90%, 95%, 97%, 98%, 99%, or more, of the biological activity of the native polypeptide (and can even have a higher level of activity than the native polypeptide). A “non-functional” polypeptide is one that exhibits essentially no detectable biological activity normally associated with the polypeptide (e.g., at most, only an insignificant amount, e.g., less than about 10% or even 5%).

A “recombinant” nucleic acid is one that has been created using genetic engineering techniques.

A “recombinant polypeptide” is one that is produced from a recombinant nucleic acid.

As used herein, an “isolated” nucleic acid (e.g., an “isolated DNA” or an “isolated vector genome”) means a nucleic acid separated or substantially free from at least some of the other components of the naturally occurring organism or virus, such as for example, the cell or viral structural components or other polypeptides or nucleic acids commonly found associated with the nucleic acid.

Likewise, an “isolated” polypeptide means a polypeptide that is separated or substantially free from at least some of the other components of the naturally occurring organism or virus, for example, the cell or viral structural components or other polypeptides or nucleic acids commonly found associated with the polypeptide. As used herein, the “isolated” polypeptide is at least about 25%, 50%, 60%, 70%, 75%, 80%, 85%, 90%, 95%, 97%, 98%, 99% or more pure (w/w).

By the term “express” or “expression” of a nucleic acid coding sequence, in particular a sequence encoding a carboxyl terminal deleted G_{M} subunit, it is meant that the sequence is transcribed, and optionally, translated.

The term “about,” as used herein when referring to a measurable value such as an amount of virus (e.g., titer), dose (e.g., an amount of a compound of the invention), time, temperature, and the like, is meant to encompass variations of ±20%, ±10%, ±5%, ±1%, ±0.5%, or even ±0.1% of the specified amount.

II. Carboxyl Terminal Deleted G_{M} Targeting Subunits.

Protein phosphatase-1 (PP-1) is activated by insulin and catalyzes the dephosphorylation of glycogen synthase and glycogen phosphorylase, thereby promoting glycogen deposition. PP-1 is targeted to the glycogen particle by a family of at least four PP-1 glycogen-targeting subunits (e.g., PP-1R6, PTG, G_{L}, G_{M}). G_{L} is expressed primarily in liver, G_{M} primarily in striated muscle, and PTG and PPP-1R6 are expressed in many tissues.

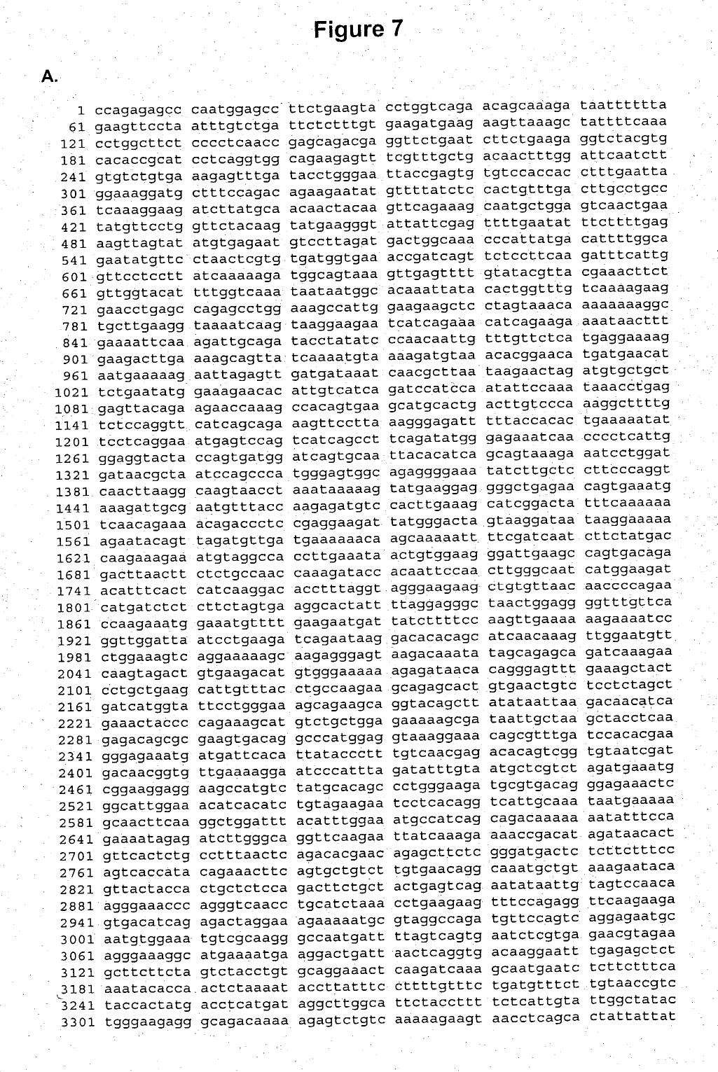

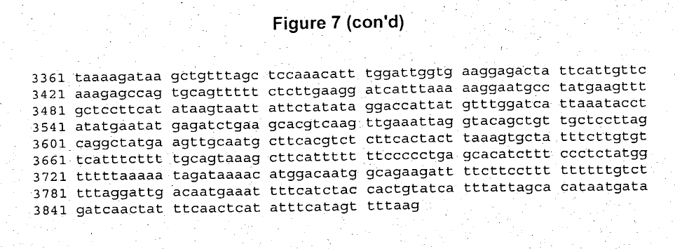

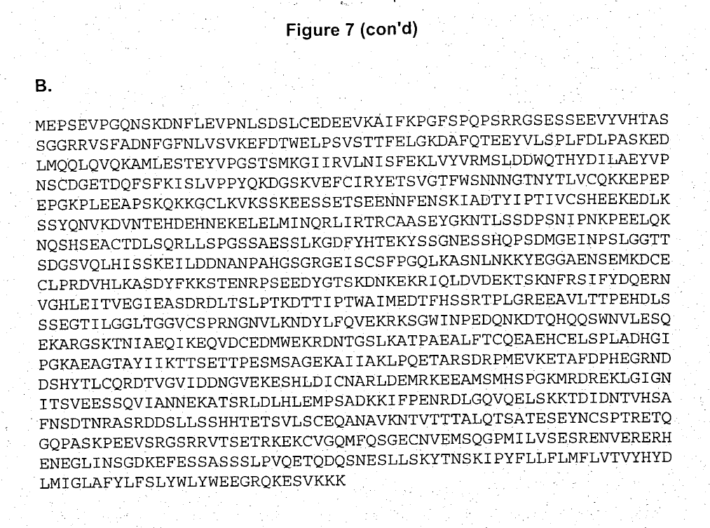

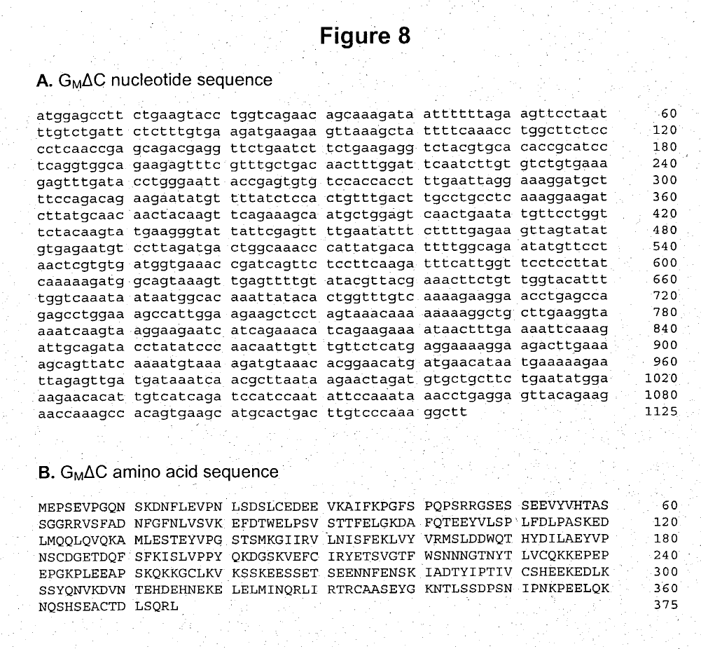

The skeletal muscle glycogen-targeting subunit G_{M} is alternatively known by the designation R_{G1} or PPP-1R3. It differs from the other glycogen targeting subunits in that it is approximately three-times as large, having a carboxyl terminal extension (e.g., amino acids 285-1122 of SEQ ID NO:8; FIG. 7). This carboxyl-terminal region includes a hydrophobic transmembrane domain that is thought to be involved in binding to the sarcoplasmic reticulum. G_{M} also contains two serines (known as “site 1” and “site 2”) that are phosphorylated in response to glycogenolytic agents. For a more detailed discussion of the PP-1 glycogen-targeting subunits, see Newgard et al., Diabetes 49:1967 (2000).

Previous studies have demonstrated, that when expressed in hepatocytes, the targeting subunits stimulate glycogen synthesis in the rank order G_{L}>PTG>G_{M}ΔC>G_{M} (Yang et al., (2002) J. Biol. Chem. 277:1514; Gasa et al., (2002) J. Biol. Chem. 277:1524). Surprisingly, G_{M}ΔC, but not G_{L} or G_{M}, ameliorate glucose intolerance when expressed in liver of rats fed on a high fat diet (Gasa et al., (2002) J. Biol. Chem. 277:1524). This appears to be explained by the finding that liver cells with overexpressed G_{M}ΔC maintain full responsiveness to glycogenolytic signals such as forskolin and low glucose, unlike cells with overexpressed G_{L} or PTG, which have impaired responsiveness to these agents, resulting in accumulation of large amounts of glycogen in the fasted state (Yang et al., (2002) J. Biol. Chem. 277:1514; Gasa et al., (2002) J. Biol. Chem. 277:1524; O'Doherty et al., (2000) J. Clin. Invest. 105:479).

The current studies investigated the properties of G_{M}ΔC in a stringent model of diabetes by expressing G_{M}ΔC in the liver of rats with streptozotocin (STZ)-induced diabetes. Three surprising observations have been made as a result of this work. First, G_{M}ΔC expression in liver is sufficient to lower blood glucose levels and raise liver glycogen levels to normal in STZ-induced diabetic rats. Second, these corrective effects on hepatic glycogen metabolism and glucose homeostasis occur despite very low levels of liver glucokinase expression in the insulin-deficient animals. Finally, hepatic G_{M}ΔC expression reduces food intake to normal levels in STZ-diabetic rats, which are otherwise hyperphagic.

As used herein, a “carboxyl terminal deleted G_{M}” refers to a PP-1 glycogen-targeting G_{M} subunit having a deletion or truncation in the unique carboxyl terminal region (e.g., the region from amino acids 285 to 1122 in FIG. 7; SEQ ID NO:8). In embodiments of the invention, the carboxyl terminal deleted G_{M} subunit enhances glycogen synthesis but does not substantially impair responsiveness to normal glycogenolytic signals as compared with the native G_{M} subunit. The carboxyl terminal deleted G_{M} subunit may be derived from the G_{M} subunit of any species, a number of which are known in the art and have been cloned and the sequences described, see, e.g., human (GenBank Accession numbers AF024576 and AH005780), mouse (GenBank Accession number NM080464) and rabbit (GenBank accession number M65109); the disclosures of which are incorporated by reference herein in their entireties.

In particular embodiments, all or a portion of the hydrophobic domain that binds to the sarcoplasmic reticulum is deleted (e.g., amino acids 1063 to 1097 in SEQ ID NO:8, see FIG. 7). The deletion can comprise at least about 5, 10, 20, 30, 50, 75, 100, 125, 150, 175, 200, 250, 300, 350, 350, 400, 450, 500, 550, 600, or 700 amino acids or more. In particular embodiments, substantially all (i.e., at least about 95% or more) of the carboxyl-terminal region is deleted.

Deletions of the carboxyl-terminal portion of the G_{M} subunit encompass carboxyl-terminal truncations. In particular embodiments, at least the carboxyl-terminal 10, 20, 30, 50, 75, 100, 125, 150, 175, 200, 250, 300, 350, 350, 400, 450, 500, 550, 600 or 700 amino acids or more are deleted from the carboxyl terminal deleted G_{M} subunit (i.e., the protein truncated at the carboxyl terminal end).

In representative embodiments, the carboxyl-terminal deleted G_{M} subunit is derived from the full-length amino acid sequence of SEQ ID NO:8 (FIG. 7). In other embodiments, the carboxyl-terminal deleted G_{M} has the amino acid sequence of SEQ ID NO:10.

In still other illustrative embodiments, the carboxyl terminal deleted mutation has a point mutation that abolishes one or both of the site 1 and site 2 serines (e.g., mutations at amino acid 48 and amino acid 67 of SEQ ID NO:8; FIG. 7, for example Ser→Ala mutations).

In representative embodiments of the invention, an isolated nucleic acid encoding a carboxyl terminal deleted G_{M} subunit will hybridize to the nucleic acid sequences specifically disclosed herein (e.g., SEQ ID NO:7 and SEQ ID NO:9) under standard conditions as known by those skilled in the art and encode a carboxyl terminal deleted G_{M} subunit that enhances glycogen synthesis without substantially impairing responsiveness to glycogenolytic signals as compared with the native protein.

To illustrate, hybridization of such sequences can be carried out under conditions of reduced stringency, medium stringency or even stringent conditions (e.g., conditions represented by a wash stringency of 35-40% Formamide with 5× Denhardt's solution, 0.5% SDS and 1×SSPE at 37° C.; conditions represented by a wash stringency of 40-45% Formamide with 5× Denhardt's solution, 0.5% SDS, and 1×SSPE at 42° C.; and/or conditions represented by a wash stringency of 50% Formamide with 5× Denhardt's solution, 0.5% SDS and 1×SSPE at 42° C., respectively) to the sequences specifically disclosed herein. See, e.g., Sambrook et al., Molecular Cloning, A Laboratory Manual (2d Ed. 1989) (Cold Spring Harbor Laboratory).

Alternatively stated, isolated nucleic acids encoding a carboxyl terminal deleted G_{M} subunit according to the present invention have at least about 60%, 70%, 80%, 90%, 95%, 97%, 98% or higher sequence similarity with the isolated nucleic acid sequences specifically disclosed herein (or fragments thereof) and encode a carboxyl terminal deleted G_{M} subunit that provides enhanced glycogen synthesis as compared with the native protein without substantially impairing responsiveness to glycogenolytic signals as compared with the native protein.

It will be appreciated by those skilled in the art that there can be variability in the nucleic acids that encode the carboxyl terminal deleted G_{M} subunit due to the degeneracy of the genetic code. The degeneracy of the genetic code, which allows different nucleic acid sequences to code for the same polypeptide, is well known in the literature (see Table 1).

TABLE 1

| | |
|---|---|
| Amino Acids | Codons |

| | | | |
|---|---|---|---|
| Alanine | Ala | A | GCA GCC GCG GCT |
| Cysteine | Cys | C | TGC TGT |
| Aspartic acid | Asp | D | GAC GAT |
| Glutamic acid | Glu | E | GAA GAG |
| Phenylalanine | Phe | F | TTC TTT |
| Glycine | Gly | G | GGA GGC GGG GGT |
| Histidine | His | H | CAC CAT |
| Isoleucine | Ile | I | ATA ATC ATT |
| Lysine | Lys | K | AAA AAG |
| Leucine | Leu | L | TTA TTG CTA CTC CTG CTT |
| Methionine | Met | M | ATG |
| Asparagine | Asn | N | AAC AAT |
| Proline | Pro | P | CCA CCC CCG CCT |
| Glutamine | Gln | Q | CAA CAG |
| Arginine | Arg | R | AGA AGG CGA CGC CGG CGT |
| Serine | Ser | S | AGC ACT TCA TCC TCG TCT |
| Threonine | Thr | T | ACA ACC ACG ACT |
| Valine | Val | V | GTA GTC GTG GTT |
| Tryptophan | Trp | W | TGG |
| Tyrosine | Tyr | Y | TAC TAT |

Further variation in the nucleic acid sequence can be introduced by the presence (or absence) of non-translated sequences, such as intronic sequences and 5′ and 3′ untranslated sequences.

Moreover, the isolated nucleic acids encoding a carboxyl terminal deleted G_{M} subunit encompass those nucleic acids encoding carboxyl terminal deleted G_{M} subunits that have at least about 60%, 70%, 80%, 90%, 95%, 97%, 98% or higher amino acid sequence similarity with the polypeptide sequences specifically disclosed herein (or fragments thereof), e.g., SEQ ID NO:8 and SEQ ID NO:10 and further encode a carboxyl terminal deleted G_{M} subunit that enhances glycogen synthesis without substantially impairing responsiveness to glycogenolytic signals as compared with the native protein.

As is known in the art, a number of different programs can be used to identify whether a nucleic acid or polypeptide has sequence identity or similarity to a known sequence. Sequence identity and/or similarity can be determined using standard techniques known in the art, including, but not limited to, the local sequence identity algorithm of Smith & Waterman, Adv. Appl. Math. 2, 482 (1981), by the sequence identity alignment algorithm of Needleman & Wunsch, J. Mol. Biol. 48,443 (1970), by the search for similarity method of Pearson & Lipman, Proc. Natl. Acad. Sci. USA 85,2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12, 387-395 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35, 351-360 (1987); the method is similar to that described by Higgins & Sharp, CABIOS 5, 151-153 (1989).

Another example of a useful algorithm is the BLAST algorithm, described in Altschul et al., J. Mol. Biol. 215, 403-410, (1990) and Karlin et al., Proc. Natl. Acad. Sci. USA 90, 5873-5787 (1993). A particularly useful BLAST program is the WU-BLAST-2 program which was obtained from Altschul et al., Methods in Enzymology, 266, 460-480 (1996); http://blast.wustl/edu/blast/README.html. WU-BLAST-2 uses several search parameters, which are preferably set to the default values. The parameters are dynamic values and are established by the program itself depending upon the composition of the particular sequence and composition of the particular database against which the sequence of interest is being searched; however, the values can be adjusted to increase sensitivity.

An additional useful algorithm is gapped BLAST as reported by Altschul et al., (1997) Nucleic Acids Res. 25, 3389-3402.

A percentage amino acid sequence identity value can be determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

In a similar manner, percent nucleic acid sequence identity with respect to the sequences disclosed herein is defined as the percentage of nucleotide residues in the candidate sequence that are identical with the nucleotides in the sequences specifically disclosed herein.

The alignment can include the introduction of gaps in the sequences to be aligned. In addition, for sequences which contain either more or fewer amino acids than the polypeptides specifically disclosed herein, it is understood that in one embodiment, the percentage of sequence identity will be determined based on the number of identical amino acids in relation to the total number of amino acids. Thus, for example, sequence identity of sequences shorter than a sequence specifically disclosed herein, will be determined using the number of amino acids in the shorter sequence. In percent identity calculations relative weight is not assigned to various manifestations of sequence variation, such as, insertions, deletions, substitutions, etc.

In one embodiment, only identities are scored positively (+1) and all forms of sequence variation including gaps are assigned a value of “0”, which obviates the need for a weighted scale or parameters as described below for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

To modify the carboxyl terminal deleted G_{M} amino acid sequences specifically disclosed herein or otherwise known in the art, amino acid substitutions can be based on any characteristic known in the art, including the relative similarity or differences of the amino acid side-chain substituents, for example, their hydrophobicity, hydrophilicity, charge, size, and the like. In particular embodiments, conservative substitutions (i.e., substitution with an amino acid residue having similar properties) are made in the amino acid sequence encoding the carboxyl terminal deleted G_{M} subunit.

In making amino acid substitutions, the hydropathic index of amino acids can be considered. The importance of the hydropathic amino acid index in conferring interactive biologic function on a protein is generally understood in the art (see, Kyte and Doolittle, (1982) J. Mol. Biol. 157:105; incorporated herein by reference in its entirety). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of its hydrophobicity and charge characteristics (Kyte and Doolittle, Id.), and these are:

isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is also understood in the art that the substitution of amino acids can be made on the basis of hydrophilicity. U.S. Pat. No. 4,554,101 (incorporated herein by reference in its entirety) states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (±3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

Isolated nucleic acids of this invention include RNA, DNA (including cDNAs) and chimeras thereof. The isolated nucleic acids can further comprise modified nucleotides or nucleotide analogs.

Those skilled in the art will appreciate that the isolated nucleic acids encoding a carboxyl terminal deleted G_{M} subunit will typically be associated with appropriate expression control sequences, e.g., transcription/translation control signals and polyadenylation signals.

It will further be appreciated that a variety of promoter/enhancer elements can be used depending on the level and tissue-specific expression desired. The promoter can be constitutive or inducible, depending on the pattern of expression desired. The promoter can be native or foreign and can be a natural or a synthetic sequence. By foreign, it is intended that the transcriptional initiation region is not found in the wild-type host into which the transcriptional initiation region is introduced. The promoter is chosen so that it will function in the target cell(s) of interest. Representative promoters are promoters that function in skeletal muscle or liver cells. Likewise, promoters that are “specific” for these cells and tissues (i.e., only show significant activity in the specific cell or tissue type) may be used.

To illustrate, in some embodiments, a promoter that provides high-level constitutive expression is desired. Examples of such promoters include, but are not limited to, the retroviral Rous sarcoma virus (RSV) LTR promoter/enhancer, the cytomegalovirus (CMV) immediate early promoter/enhancer (see, e.g., Boshart et al, (1985) Cell 41:521), the SV40 promoter, the dihydrofolate reductase promoter, the cytoplasmic β-actin promoter and the phosphoglycerol kinase (PGK) promoter.

In other embodiments, inducible promoters are desired. Inducible promoters are those which are regulated by exogenously supplied compounds, including without limitation, the zinc-inducible metalothionein (MT) promoter; the dexamethasone (Dex)-inducible mouse mammary tumor virus (MMTV) promoter; the T7 polymerase promoter system (see WO 98/10088); the ecdysone insect promoter (No et al, (1996) Proc. Natl. Acad. Sci. USA 93:3346); the tetracycline-repressible system (Gossen et al., (1992) Proc. Natl. Acad. Sci. USA 89:5547); the tetracycline-inducible system (Gossen et al., (1995) Science 268:1766; see also Harvey et al., (1998) Curr. Opin. Chem. Biol. 2:512); the RU486-inducible system (Wang et al., (1997) Nat. Biotech. 15:239; Wang et al., (1997) Gene Ther., 4:432); and the rapamycin-inducible system (Magari et al., (1997) J. Clin. Invest. 100:2865). Other types of inducible promoters that may be useful in this context are those which are regulated by a specific physiological state, e.g., temperature, acute phase, or in replicating cells only.

In another embodiment of the invention, the promoter is operable in a particular tissue of interest. For instance, in representative embodiments, promoters that are operable in skeletal muscle or liver are desired. Promoters that function in skeletal muscle include promoters from genes encoding skeletal α-actin, myosin light chain 2A, dystrophin, muscle creatine kinase, as well as synthetic muscle promoters with activities higher than naturally-occurring promoters (see, Li et al., (1999) Nat. Biotech. 17:241). Examples of promoters that function in liver include, without limitation, the albumin promoter (Miyatake et al., (1997) J. Virol. 71:5124); hepatitis B virus core promoter (Sandig et al., (1996) Gene Ther. 3:1002); the hepatitis B X-gene promoter, alpha-fetoprotein promoter (AFP; Arbuthnot et al., (1996) Hum. Gene Ther. 7:1503), alpha-1 antitrypsin gene promoter, the apolipoprotein A1 promoter, and promoters for liver enzymes such as, for example, SGOT, SGPT and gamma-glutamyl transference.

Moreover, specific initiation signals are generally required for efficient translation of inserted protein coding sequences. These translational control sequences, which can include the ATG initiation codon and adjacent sequences, can be of a variety of origins, both natural and synthetic.

The isolated nucleic acids encoding the carboxyl terminal deleted G_{M} subunit can be incorporated into a vector, e.g., for the purposes of cloning or other laboratory manipulations, recombinant protein production, or gene delivery. Exemplary vectors include bacterial artificial chromosomes, cosmids, yeast artificial chromosomes, phage, plasmids, lipid vectors and viral vectors (described in more detail below). Nucleic acid delivery vectors are more specifically described in Section V.

The present invention further provides cells comprising the carboxyl terminal deleted G_{M} subunit and/or the isolated nucleic acids encoding the same. Such cells find use, for example, in the screening methods of the invention and in ex vivo gene delivery protocols.

III. Screening Assays.

The finding that modulation of G_{M} targeting subunit activity can reduce hyperglycemia and caloric intake (e.g., reduce hyperphagia) provides new targets for treating these conditions. Likewise, the discovery that cellular function can be modulated to enhance glycogen synthesis without substantially impairing responsiveness to glycogenolytic signals (i.e., regulation of glycogen synthesis/breakdown can be maintained) opens up new approaches for identifying compounds to treat hyperglycemia and/or to reduce caloric intake (e.g., induce satiety, reduce hyperphagia and/or reduce appetite).

Accordingly, in one aspect, the present invention provides methods of identifying a compound or compounds that enhance glycogen synthesis without substantially impairing responsiveness to glycogenolytic signals (e.g., in hepatic or skeletal muscle cells). In particular embodiments, evaluation of glycogen synthesis and glycogen breakdown are carried out in a substantially concurrent manner. In other embodiments, these steps are carried out sequentially, e.g., a compound may first be identified as enhancing glycogen synthesis and then be evaluated for its effects on glycogen breakdown. According to this embodiment, there may be a significant time span between assessing the effects of the compound on glycogen synthesis and the effects on glycogenolysis, e.g., because of intervening studies to further characterize the compound or to identify a pool of compounds to take to the next step of evaluation.

As another aspect, the present invention provides a method of identifying a compound that can reduce caloric intake, comprising: contacting a cell with a compound under conditions whereby glycogen synthesis and glycogenolysis can be detected; and detecting whether the compound enhances glycogen synthesis but does not substantially impair responsiveness to glycogenolytic signals, thereby identifying a compound that can reduce caloric intake.

As a further aspect, the invention provides a method of identifying a compound that can reduce hyperglycemia, comprising; contacting a cell with a compound under conditions whereby glycogen synthesis and glycogenolysis can be detected; and detecting whether the compound enhances glycogen synthesis but does not substantially impair responsiveness to glycogenolytic signals, thereby identifying a compound that can reduce hyperglycemia.

Any desired end-point can be detected to assess glycogen synthesis and/or glycogen breakdown, e.g., glycogen stores, glucose concentrations in blood, serum or culture medium, abundance and/or activity of glycogenic or glycogenolytic enzymes, and the like.

In another representative embodiment, the invention provides methods of screening test compounds to identify a compound that binds to a G_{M} subunit or a carboxyl terminal deleted G_{M} subunit. Compounds that are identified as binding to the native or carboxyl terminal deleted G_{M} can be subject to further screening using the methods described herein (e.g., for enhancement of the activity of the protein, enhancement of glycogen synthesis, and/or for effects on glycogen breakdown) or other suitable techniques.

Also provided are methods of identifying compounds that bind to and/or enhance the activity of PP-1 or glycogen synthase. Methods of measuring the abundance of PP-1 or glycogen synthase protein (e.g., using standard methods of detecting proteins, such as Western blots or methods based on immunological detection) or the activity of these enzymes are known in the art (see, e.g., Gasa et al., (2000) J. Biol. Chem. 275:26396). The compound may directly bind to PP-1 or glycogen synthase to enhance the activity thereof. Alternatively, the compound can interact with the DNA or RNA sequences encoding PP-1 or glycogen synthase to increase production of the polypeptide. As a further alternative, the compound can interact with any other polypeptide, nucleic acid or other molecule as long as the interaction results in an enhancement of PP-1 or glycogen synthase activity.

Alternatively, the method comprises a method of identifying compounds that bind to and/or inhibit the activity of glycogen phosphorylase.

Any compound of interest can be screened according to the present invention. Suitable test compounds include organic and inorganic molecules. Organic molecules can include but are not limited to polypeptides (including enzymes, antibodies and Fab′ fragments), carbohydrates, lipids, coenzymes, and nucleic acid molecules (including DNA, RNA and chimerics and analogs thereof). In particular embodiments, the compound is an antisense nucleic acid, an interfering RNA (RNAi), a ribozyme or any other molecule that results in enhanced glycogen synthesis without substantial impairment in the response to glycogenolytic signals.

Further, the methods of the invention can be practiced to screen a compound library, e.g., a combinatorial chemical compound library, a polypeptide library, a cDNA library, a library of antisense nucleic acids, and the like, or an arrayed collection of compounds such as polypeptide and nucleic acid arrays.

The foregoing screening methods can be based on cell-based or cell-free assays or, alternatively, can be carried out in an intact animal. Cell based assays also encompass assays that display a target polypeptide on the surface of a cell. In the case of cell-free assays, the target polypeptide (as described above) can be free in solution, affixed to a solid support, and the like. For cell-based methods or methods using an intact animal, the target polypeptide can be endogenously produced by the cell or the animal. Alternatively or additionally, the cell or animal can be genetically modified to comprise an isolated nucleic acid encoding, and optionally overexpressing, the target polypeptide. According to this embodiment, the cell or animal can be transiently or stably transformed with the nucleic acid encoding the target polypeptide, but is typically stably transformed, for example, by stable integration into the genome of the organism or by expression from a stably maintained episome (e.g., Epstein Barr Virus derived episomes).

With respect to cell-based assays, any suitable cell can be used including bacteria, yeast, insect cells (e.g., with a baculovirus expression system), avian cells, mammalian cells, or plant cells. In particular embodiments, the cells are from a diabetic subject or other subject with insulin resistance, hypoglycemia, an obese subject, and/or a subject with hyperphagia. In other representative embodiments, the cell has low glucokinase activity.

The cell may have low glucokinase activity in its native state (including low glucokinase activity as the result of a disease state or other condition, e.g., MODY-2 or STZ treatment). Alternatively, glucokinase activity in the cell can be reduced by a variety of methods known in the art, e.g., antisense oligonucleotides, RNAi, targeted disruption of the glucokinase coding sequence in the cell, selection for cells that have low glucokinase activity, and the like.

Cell based and whole animal approaches are suitable for methods in which the end-point to be evaluated is glycogen synthesis and/or glycogen breakdown. These methods can be carried out with any cell (or tissue) that can synthesize glycogen. Such cells include primary hepatic cells, hepatoma cell lines, primary skeletal muscle cells, muscle cell lines, and any other cell that can synthesize glycogen (including cells that have been genetically modified to confer glycogen synthesis capability). As described above, the subject can be a subject with diabetes, hyperglycemia or other insulin resistant condition, an obese subject and/or a subject with hyperphagia (or the cell can be derived from such a subject). Likewise, subject can have low glucokinase levels (e.g., in the liver), or the cell can be derived from a subject with low glucokinase levels.

As a further type of cell-based binding assay, the target polypeptide of interest can be used as a “bait protein” in a two-hybrid or three-hybrid assay (see, e.g., U.S. Pat. No. 5,283,317; Zervos et al., (1993) Cell 72:223-232; Madura et al., (1993) J. Biol. Chem. 268:12046-12054; Bartel et al., (1993) Biotechniques 14:920-924; Iwabuchi et al., (1993) Oncogene 8:1693-1696; and PCT publication WO94/10300), to identify other polypeptides that bind to or interact with the target polypeptide.

The two-hybrid system is based on the modular nature of most transcription factors, which consist of separable DNA-binding and activation domains. Briefly, the assay utilizes two different DNA constructs. In one construct, the nucleic acid that encodes a target polypeptide is fused to a nucleic acid encoding the DNA binding domain of a known transcription factor (e.g., GAL-4). In the other construct, a DNA sequence, optionally from a library of DNA sequences, that encodes an unidentified protein (“prey” or “sample”) is fused to a nucleic acid that codes for the activation domain of the known transcription factor. If the “bait” and the “prey” proteins are able to interact in vivo, forming a complex, the DNA-binding and activation domains of the transcription factor are brought into close proximity. This proximity allows transcription of a reporter sequence (e.g., LacZ), which is operably linked to a transcriptional regulatory site responsive to the transcription factor. Expression of the reporter can be detected and cell colonies containing the functional transcription factor can be isolated and used to obtain the nucleic acid encoding the polypeptide that exhibited binding to the target polypeptide.

In cell-free binding assays, test compounds can be synthesized or otherwise affixed to a solid substrate, such as plastic pins, glass slides, plastic wells, and the like. For example, the test compounds can be immobilized utilizing conjugation of biotin and streptavidin by techniques well known in the art. The test compounds are contacted with the target polypeptide and washed. The bound polypeptide can be detected using standard techniques in the art (e.g., by radioactive or fluorescence labeling of the target polypeptide, by ELISA methods, and the like).

Alternatively, the target polypeptide can be immobilized to a solid substrate and the test compounds contacted with the bound polypeptide. Identifying those test compounds that bind to and/or modulate the activity of the polypeptide can be carried out with routine techniques. For example, the test compounds can be immobilized utilizing conjugation of biotin and streptavidin by techniques well known in the art. As another illustrative example, antibodies reactive with the polypeptide can be bound to the wells of the plate, and the polypeptide trapped in the wells by antibody conjugation. Preparations of test compounds can be incubated in the polypeptide-presenting wells and the amount of complex trapped in the well can be quantitated.

In another representative embodiment, a fusion protein can be provided which comprises a domain that facilitates binding of the protein to a matrix. For example, glutathione-S-transferase fusion proteins can be adsorbed onto glutathione sepharose beads (Sigma Chemical, St. Louis, Mo.) or glutathione derivatized microtitre plates, which are then combined with cell lysates (e.g., ³⁵S-labeled) and the test compound, and the mixture incubated under conditions conducive to complex formation (e.g., at physiological conditions for salt and pH). Following incubation, the beads are washed to remove any unbound label, and the matrix immobilized and radiolabel detected directly, or in the supernatant after the complexes are dissociated. Alternatively, the complexes can be dissociated from the matrix, separated by SDS-PAGE, and the level of polypeptide found in the bead fraction quantitated from the gel using standard electrophoretic techniques.

Another technique provides for high throughput screening of compounds having suitable binding affinity to the polypeptide of interest, as described in published PCT application WO84/03564. In this method, a large number of different small test compounds are synthesized on a solid substrate, such as plastic pins or some other surface. The test compounds are reacted with the target polypeptide and washed. Bound polypeptide is then detected by methods well known in the art. The purified target polypeptide can also be coated directly onto plates for use in the aforementioned drug screening techniques. Alternatively, non-neutralizing antibodies can be used to capture the peptide and immobilize it on a solid support.

Screening assays can also be carried out in vivo in animals. The animal can be of any species, including avians and mammals. According to this aspect of the invention, suitable mammals include mice, rats, rabbits, guinea pigs, goats, sheep, pigs, cattle, primate (including humans). Mammalian models for glucose intolerance, obesity, hyperglycemia, diabetes, hyperphagia and low glucokinase can also be used (e.g., STZ diabetic mice, ob/ob mice). Suitable avians include chickens, ducks, geese, quail, turkeys and pheasants.

In particular embodiments, the target polypeptide is endogenous to the animal. In other embodiments, a nucleic acid encoding a target polypeptide will be administered to an animal subject to provide transient or stable expression of the encoded polypeptide. Typically, stable incorporation of the nucleic acid into the genome (e.g., by stable integration into the genome or by stably maintained episomal constructs) is desirable. It is not necessary that every cell contain the transgene, and the animal can be a chimera of modified and unmodified cells, as long as a sufficient number of glycogen synthesizing cells incorporate the nucleic acid and produce the target polypeptide so that the animal is a useful screening tool (e.g., so that enhancement of glycogen synthesis or impairment of glycogen breakdown in response to glycogenolytic signals can be detected).

In particular embodiments, the invention provides a method of administering a compound to a subject and detecting whether the compound enhances the activity of a G_{M} subunit, PP-1, glycogen synthase, and/or any other target polypeptide of interest. Alternatively, the method can comprise a method of administering a compound that inhibits glycogen phosphorylase.

In another particular embodiment, the invention provides a method of administering a compound to a subject and detecting whether the compound enhances glycogen synthesis but does not substantially impair responsiveness to glycogenolytic signals (e.g., in the liver or skeletal muscle).

As still another representative embodiment, the invention provides a method of identifying a compound that can reduce caloric intake, comprising: administering a compound to a subject under conditions whereby glycogen synthesis and glycogenolysis can be detected; and detecting whether the compound enhances glycogen synthesis but does not substantially impair responsiveness to glycogenolytic signals, thereby identifying a compound that can reduce caloric intake.

As a further illustrative embodiment, the invention provides a method of identifying a compound that can reduce hyperglycemia, comprising: administering a compound to a subject under conditions whereby glycogen synthesis and glycogenolysis can be detected; and detecting whether the compound enhances glycogen synthesis but does not substantially impair responsiveness to glycogenolytic signals, thereby identifying a compound that can reduce hyperglycemia.

As described above, glycogen synthesis and glycogen breakdown can be evaluated concurrently, sequentially or even with a significant intervening gap in time.

Methods of making transgenic animals are known in the art. DNA constructs can be introduced into the germ line of an avian or mammal to make a transgenic animal. For example, one or several copies of the construct can be incorporated into the genome of an embryo by standard transgenic techniques.

In an exemplary embodiment, a transgenic animal is produced by introducing a transgene into the germ line of the animal. Transgenes can be introduced into embryonal target cells at various developmental stages. Different methods are used depending on the stage of development of the embryonal target cell. The specific line(s) of any animal used should, if possible, be selected for general good health, good embryo yields, good pronuclear visibility in the embryo, and good reproductive fitness.

Introduction of the transgene into the embryo can be accomplished by any of a variety of means known in the art such as microinjection, electroporation, lipofection or a viral vector. For example, the transgene can be introduced into a mammal by microinjection of the construct into the pronuclei of the fertilized mammalian egg(s) to cause one or more copies of the construct to be retained in the cells of the developing mammal(s). Following introduction of the transgene construct into the fertilized egg, the egg can be incubated in vitro for varying amounts of time, or reimplanted into the surrogate host, or both. One common method is to incubate the embryos in vitro for about 1-7 days, depending on the species, and then reimplant them into the surrogate host.

The progeny of the transgenically manipulated embryos can be tested for the presence of the construct by Southern blot analysis of a segment of tissue. An embryo having one or more copies of the exogenous cloned construct stably integrated into the genome can be used to establish a permanent transgenic animal line carrying the transgenically added construct.

Transgenically altered animals can be assayed after birth for the incorporation of the construct into the genome of the offspring. This can be done by hybridizing a probe corresponding to the DNA sequence coding for the polypeptide or a segment thereof onto chromosomal material from the progeny. Those progeny found to contain at least one copy of the construct in their genome are grown to maturity.

Methods of producing transgenic avians are also known in the art, see, e.g., U.S. Pat. No. 5,162,215.

It will be appreciated by those skilled in the art that the screening methods of the invention can be used together in any combination.

IV. Compounds and Methods for Enhancing Glycogen Synthesis While Maintaining Normal Regulation by Glycogenolytic Signals.

The investigations described herein demonstrate that enhancing glycogen synthesis can substantially contribute to normalization of blood glucose concentrations in diabetic subjects. Moreover, overexpression of full-length PTG and G_{L} subunits results in a condition resembling a glycogen storage disease, i.e., increased glycogen deposition was observed, but responsiveness to glycogenolytic signals such as hypoglycemia was impaired. Further, overexpression of G_{M} subunit was found to be non-efficacious in lowering blood glucose concentrations. In contrast, expression of a carboxyl terminal deleted G_{M} subunit resulted in enhanced glycogen synthesis and normalization of blood glucose concentrations without substantially impairing responsiveness to regulatory signals that trigger glycogen breakdown. Strikingly, these effects are observed in the presence of low glucokinase activity, indicating that glycogen synthesis can be regulated at a site distal to glucokinase. Furthermore, the inventors have found that expression of a carboxyl terminal deleted G_{M} subunit reduced caloric intake in hyperphagic animals. These investigations redefine current models of glycogenesis and point to new approaches and targets for regulating glycogenesis, blood glucose concentrations, and caloric intake. These studies also demonstrate that glycogen synthesis can be enhanced without losing homeostatic control over the balance between glycogen synthesis and glycogen breakdown.

As one aspect, the present invention provides methods of administering a nucleic acid encoding a carboxyl terminal deleted G_{M} to enhance glycogen synthesis without substantially impairing responsiveness to glycogenolytic signals. Now that the inventors have demonstrated that cellular function may be modulated to achieve these effects, the present invention further encompasses methods of administering other compounds (the compounds of the invention are described in more detail below) that mimic the effects of a carboxyl terminal deleted G_{M} on glycogenesis/glycogenolysis.

The compound can interact directly with a G_{M} targeting subunit or the coding sequence for a G_{M} targeting subunit to modulate the activity thereof.

The compounds of the invention can bind directly to the G_{M} subunit and enhance the activity thereof, without substantially impairing responsiveness of the cell (organ, subject) to glycogenolytic signals. Compounds that specifically bind to the G_{M} subunit can bind to the carboxyl terminal portion, to the glycogen binding domain, to the hydrophobic transmembrane domain, to the site 1 and/or site 2 phosphorylation sites, to the PP-1 binding domain or elsewhere, and can further alter binding of the G_{M} subunit to glycogen and/or PP-1.

Alternatively, the compound can interact with any other polypeptide, nucleic acid or other molecule if such interaction results in a enhancement of glycogen synthesis without substantially impairing responsiveness to glycogenolytic signals (e.g., in the liver or skeletal muscle). In representative embodiments, the compound interacts with PP-1 or glycogen synthase to enhance the activity thereof (and thereby enhance glycogen synthesis) without substantially impairing responsiveness to glycogenolytic signals. As another alternative, the compound can interact with another polypeptide or nucleic acid to inhibit the activity thereof, if such interaction results in an enhancement in glycogen synthesis without substantial impairment of responsiveness to glycogenolytic signals. As still another alternative, the compound can be a glycogen phosphorylase. For example, as described in U.S. Pat. Nos. 6,297,269; 6,277,877; 6,107,329 and 5,952,322 (the disclosures of which are incorporated herein by reference in their entirety for teaching of compounds useful as glycogen phosphorylase inhibitors) disclose substituted n-(indole-2-carbonyl-) amides, substituted n-(indole-2-carbonyl-) glycinamides, and derivatives of the foregoing as glycogen phosphorylase inhibitors. Such compounds can be used according to the methods of the present invention, e.g., to reduce hyperglycemia or to reduce caloric intake (e.g., to treat hyperphagia, induce satiety and/or to reduce appetite).

Non-limiting examples of compounds that are glycogen phosphorylase inhibitors and that may be used to carry out the present invention are set forth below:

One group of glycogen phosphorylase inhibitors includes compounds of the Formula I

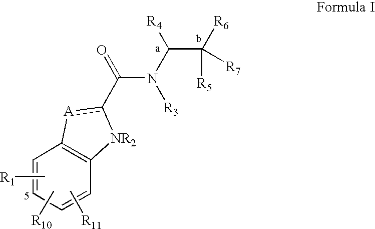

and the pharmaceutically acceptable salts and prodrugs thereof wherein

the dotted line (—) is an optional bond;

A is —C(H)═, —C((C₁-C₄)alkyl)=or —C(halo)=when the dotted line (—) is a bond, or A is methylene or —CH((C₁-C₄)alkyl)- when the dotted line (—) is not a bond;

R₁, R₁₀ or R₁₁, are each independently H, halo, 4-, 6- or 7-nitro, cyano, (C₁-C₄)alkyl, (C₁-C₄)alkoxy, fluoromethyl, difluoromethyl or trifluoromethyl;

R₂ is H;

R₃ is H or (C₁-C₅)alkyl;

R₄ is H, methyl, ethyl, n-propyl, hydroxy(C₁-C₃)alkyl, (C₁-C₃)alkoxy(C₁-C₄)alkyl, phenyl(C₁-C₄)alkyl, phenylhydroxy(C₁-C₄)alkyl, phenyl(C₁-C₄)alkoxy(C₁-C₄)alkyl, thien-2- or -3-yl(C₁-C₄)alkyl or fur-2- or -3-yl(C₁-C₄)alkyl wherein said R₄ rings are mono-, di- or tri-substituted independently on carbon with H, halo, (C₁-C₄)alkyl, (C₁-C₄)alkoxy, trifluoromethyl, hydroxy, amino or cyano; or

R₄ is pyrid-2-, -3- or -4-yl(C₁-C₄)alkyl, thiazol-2-, -4- or -5-yl(C₁-C₄)alkyl, imidazol -1-, -2-, -4- or -5-yl(C₁-C₄)alkyl, pyrrol-2- or -3-yl(C₁-C₄)alkyl, oxazol-2-, -4- or -5-yl-(C₁-C₄)alkyl, pyrazol-3-, -4- or -5-yl(C₁-C₄)alkyl, isoxazol-3-, -4- or -5-yl(C₁-C₄)alkyl, isothiazol-3-, -4- or -5-yl(C₁-C₄)alkyl, pyridazin-3- or -4-yl-(C₁-C₄)alkyl, pyrimidin-2-, -4-, -5- or -6-yl(C₁-C₄)alkyl, pyrazin-2- or -3-yl(C₁-C₄)alkyl or 1,3,5-traiazin-2-yl(C₁-C₄)alkyl, wherein said preceding R₄ heterocycles are optionally mono- or di-substituted independently with halo, trifluoromethyl, (C₁-C₄)alkyl, (C₁-C₄)alkoxy, amino or hydroxy and said mono- or di-substituents are bonded to carbon;

R₅ is H, hydroxy, fluoro, (C₁-C₅)alkyl, (C₁-C₅)alkoxy, (C₁-C₆)alkanoyl, amino(C₁-C₄)alkoxy, mono-N- or di-N,N-(C₁-C₄)alkylamino(C₁-C₄)alkoxy, carboxy(C₁-C₄)alkoxy, (C₁-C₅)alkoxy-carbonyl(C₁-C₄)alkoxy, benzyloxycarbonyl(C₁-C₄)alkoxy, or carbonyloxy wherein said carbonyloxy is carbon-carbon linked with phenyl, thiazolyl, imidazolyl, 1H-indolyl, furyl, pyrrolyl, oxazolyl, pyrazolyl, isoxazolyl, isothiazolyl, pyridazinyl, pyrimidinyl, pyrazinyl or 1,3,5-triazinyl and wherein said preceding R₅ rings are optionally mono-substituted with halo, (C₁-C₄)alkyl, (C₁-C₄)alkoxy, hydroxy, amino or trifluoromethyl and said mono-substituents are bonded to carbon;

R₇ is H, fluoro or (C₁-C₅)alkyl; or

R₅ and R₇ can be taken together to be oxo;

R₆ is carboxy, (C₁-C₈)alkoxycarbonyl, C(O)NR₈R₉ or C(O)R₁₂, wherein

R₈ is H, (C₁-C₃)alkyl, hydroxy or (C₁-C₃)alkoxy; and

R₉ is H, (C₁-C₈)alkyl, hydroxy, (C₁-C₈)alkoxy, methylene-perfluorinated(C₁-C₈)alkyl, phenyl, pyridyl, thienyl, furyl, pyrrolyl, pyrrolidinyl, oxazolyl, thiazolyl, imidazolyl, pyrazolyl, pyrazolinyl, pyrazolidinyl, isoxazolyl, isothiazolyl, pyranyl, piperidinyl, morpholinyl, pyridazinyl, pyrimidinyl, pyrazinyl, piperazinyl or 1,3,5-triazinyl wherein said preceding R₉ rings are carbon-nitrogen linked; or

R₉ is mono-, di- or tri-substituted (C₁-C₅)alkyl, wherein said substituents are independently H, hydroxy, amino, mono-N- or di-N,N-(C₁-C₅)alkylamino; or

R₉ is mono- or di-substituted (C₁-C₅)alkyl, wherein said substituents are independently phenyl, pyridyl, furyl, pyrrolyl, pyrrolidinyl, oxazolyl, thiazolyl, imidazolyl, pyrazolyl, pyrazolinyl, pyrazolidinyl, isoxazolyl, isothiazolyl, pyranyl, pyridinyl, piperidinyl, morpholinyl, pyridazinyl, pyrimidinyl, pyrazinyl, piperazinyl or 1,3,5-triazinyl

wherein the nonaromatic nitrogen-containing R₉ rings are optionally mono-substituted on nitrogen with (C₁-C₆)alkyl, benzyl, benzoyl or (C₁-C₆)alkoxycarbonyl and wherein the R₉ rings are optionally mono-substituted on carbon with halo, (C₁-C₄)alkyl, (C₁-C₄)alkoxy, hydroxy, amino, or mono-N- and di-N,N(C₁-C₅)alkylamino provided that no quaternized nitrogen is included and there are no nitrogen-oxygen, nitrogen-nitrogen or nitrogen-halo bonds;

R₁₂ is piperazin-1-yl, 4-(C₁-C₄)alkylpiperazin-1-yl, 4-formylpiperazin-1-yl, morpholino, thiomorpholino, 1-oxothiomorpholino, 1,1-dioxo-thiomorpholino, thiazolidin-3-yl, 1-oxo-thiazolidin-3-yl, 1,1-dioxo-thiazolidin-3-yl, 2-(C₁-C₆)alkoxycarbonylpyrrolidin-1-yl, oxazolidin-3-yl or 2(R)-hydroxymethylpyrrolidin-1-yl; or

R₁₂ is 3- and/or 4-mono- or di-substituted oxazetidin-2-yl, 2-, 4-, and/or 5mono- or di-substituted oxazolidin-3-yl, 2-, 4-, and/or 5-mono- or di-substituted thiazolidin-3-yl, 2-, 4-, and/or 5-mono- or di-substituted 1-oxothiazolidin-3-yl, 2-, 4-, and/or 5-mono- or di-substituted 1,1-dioxothiazolidin-3-yl, 3- and/or 4-, mono- or di-substituted pyrrolidin-1-yl, 3-, 4- and/or 5-, mono-, di- or tri-substituted piperidin-1-yl, 3-, 4-, and/or 5-mono-, di-, or tri-substituted piperazin-1-yl, 3-substituted azetidin-1-yl, 4- and/or 5-, mono- or di-substituted 1,2-oxazinan-2-yl, 3- and/or 4-mono- or di-substituted pyrazolidin-1-yl, 4- and/or 5-, mono- or di-substituted isoxazolidin-2-yl, 4- and/or 5-, mono- and/or di-substituted isothiazolidin-2-yl wherein said R₁₂ substituents are independently H, halo, (C₁-C₅)-alkyl, hydroxy, amino, mono-N- or di-N,N-(C₁-C₅)alkylamino, formyl, oxo, hydroxyimino, (C₁-C₅)alkoxy, carboxy, carbamoyl, mono-N-or di-N,N-(C₁-C₄)alkylcarbamoyl, (C₁-C₄)alkoxyimino, (C₁-C₄)alkoxymethoxy, (C₁-C₆)alkoxycarbonyl, carboxy(C₁-C₅)alkyl or hydroxy(C₁-C₅)alkyl;

with the proviso that if R₄ is H, methyl, ethyl or n-propyl R₅ is OH;

with the proviso that if R₅ and R₇ are H, then R₄ is not H, methyl, ethyl, n-propyl, hydroxy(C₁-C₃)alkyl or (C₁-C₃)alkoxy(C₁-C₃)alkyl and R₆ is C(O)NR₈R₉, C(O)R₁₂ or (C₁-C₄)alkoxycarbonyl.

A second group of glycogen phosphorylase inhibitors includes compounds of the Formula IA

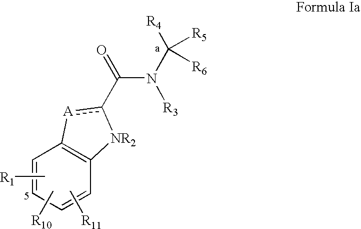

and the pharmaceutically acceptable salts and prodrugs thereof wherein

the dotted line (—) is an optional bond;

A is —C(H)═, —C((C₁-C₄)alkyl)=, —C(halo)= or —N═, when the dotted line (—) is a bond, or A is methylene or —CH((C₁-C₄)alkyl)-, when the dotted line (—) is not a bond;

R₁, R₁₀ or R₁₁ are each independently H, halo, cyano, 4-, 6-, or 7-nitro, (C₁-C₄)alkyl, (C₁-C₄)alkoxy, fluoromethyl, difluoromethyl or trifluoromethyl;

R₂ is H;

R₃ is H or (C₁-C₅)alkyl;

R₄ is H, methyl, ethyl, n-propyl, hydroxy(C₁-C₃)alkyl, (C₁-C₃)alkoxy(C₁-C₃)alkyl, phenyl(C₁-C₄)alkyl, phenylhydroxy(C₁-C₄)alkyl, (phenyl)((C₁-C₄)-alkoxy) (C₁-C₄)alkyl, thien-2- or -3-yl(C₁-C₄)alkyl or fur-2- or -3-yl(C₁-C₄)alkyl wherein said R₄ rings are mono-, di- or tri-substituted independently on carbon with H, halo, (C₁-C₄)alkyl, (C₁-C₄)alkoxy, trifluoromethyl, hydroxy, amino, cyano or 4,5-dihydro-1H-imidazol-2-yl; or

R₄ is pyrid-2-, -3- or -4-yl(C₁-C₄)alkyl, thiazol-2-, -4- or -5-yl(C₁-C₄)alkyl, imidazol-2-, -4- or -5-yl(C₁-C₄)alkyl, pyrrol-2- or -3-yl(C₁-C₄)alkyl, oxazol-2-, -4- or -5-yl(C₁-C₄)alkyl, pyrazol-3-, -4- or -5-yl(C₁-C₄)alkyl, isoxazol-3-, -4- or -5-yl(C₁-C₄)alkyl, isothiazol-3-, -4- or -5-yl(C₁-C₄)alkyl, pyridazin-3- or -4-yl(C₁-C₄)alkyl, pyrimidin-2-, -4-, -5- or -6-yl(C₁-C₄)alkyl, pyrazin-2- or -3-yl(C₁-C₄)alkyl, 1,3,5-triazin-2-yl(C₁-C₄)alkyl or indol-2-(C₁-C₄)alkyl, wherein said preceding R₄ heterocycles are optionally mono- or di-substituted independently with halo, trifluoromethyl, (C₁-C₄)alkyl, (C₁-C₄)alkoxy, amino, hydroxy or cyano and said substituents are bonded to carbon; or

R₄ is R₁₅-carbonyloxymethyl, wherein said R₁₅ is phenyl, thiazolyl, imidazolyl, 1H-indolyl, furyl, pyrrolyl, oxazolyl, pyrazolyl, isoxazolyl, isothiazolyl, pyridyl, pyridazinyl, pyrimidinyl, pyrazinyl or 1,3,5-triazinyl and wherein said preceding R₁₅ rings are optionally mono- or di-substituted independently with halo, amino, hydroxy, (C₁-C₄)alkyl, (C₁-C₄)alkoxy or trifluoromethyl and said mono- or di-substituents are bonded to carbon;

R₅ is H;

R₆ is carboxy, (C₁-C₈)alkoxycarbonyl, benzyloxycarbonyl, C(O)NR₈R₉ or C(O)R₁₂

wherein

R₈ is H, (C₁-C₆)alkyl, cyclo(C₃-C₆)alkyl, cyclo(C₃-C₆)alkyl(C₁-C₅)alkyl, hydroxy or (C₁-C₈)alkoxy; and

R₉ is H, cyclo(C₃-C₈)alkyl, cyclo(C₃-C₈)alkyl(C₁-C₅)alkyl, cyclo(C₄-C₇)alkenyl, cyclo(C₃-C₇)alkyl(C₁-C₅)alkoxy, cyclo(C₃-C₇)alkyloxy, hydroxy, methylene-perfluorinated(C₁-C₈)alkyl, phenyl, or a heterocycle wherein said heterocycle is pyridyl, furyl, pyrrolyl, pyrrolidinyl, oxazolyl, thiazolyl, imidazolyl, pyrazolyl, pyrazolinyl, pyrazolidinyl, isoxazolyl, isothiazolyl, pyranyl, pyridinyl, piperidinyl, morpholinyl, pyridazinyl, pyrimidinyl, pyrazinyl, piperazinyl, 1,3,5-triazinyl, benzothiazolyl, benzoxazolyl, benzimidazolyl, thiochromanyl or tetrahydrobenzothiazolyl wherein said heterocycle rings are carbon-nitrogen linked; or

R₉ is (C₁-C₆)alkyl or (C₁-C₈)alkoxy wherein said (C₁-C₆)alkyl or (C₁-C₈)alkoxy is optionally monosubstituted with cyclo(C₄-C₇)alken-1-yl, phenyl, thienyl, pyridyl, furyl, pyrrolyl, pyrrolidinyl, oxazolyl, thiazolyl, imidazolyl, pyrazolyl, pyrazolinyl, pyrazolidinyl, isoxazolyl, isothiazolyl, pyranyl, piperidinyl, morpholinyl, thiomorpholinyl, 1-oxothiomorpholinyl, 1,1-dioxothiomorpholinyl, pyridazinyl, pyrimidinyl, pyrazinyl, piperazinyl, 1,3,5-triazinyl or indolyl and wherein said (C₁-C₆)alkyl or (C₁-C₈)alkoxy are optionally additionally independently mono- or di-substituted with halo, hydroxy, ((C₁-C₅)alkoxy, amino, mono-N- or di-N,N-(C₁-C₅)alkylamino, cyano, carboxy, or (C₁-C₄)alkoxycarbonyl; and

wherein the R₉ rings are optionally mono- or di-substituted independently on carbon with halo, (C₁-C₄)alkyl, (C₁-C₄)alkoxy, hydroxy, hydroxy(C₁-C₄)alkyl, amino(C₁-C₄)alkyl, mono-N- or di-N,N-(C₁-C₄)alkylamino(C₁-C₄)alkyl, (C₁-C₄)alkoxy(C₁-C₄)alkyl, amino, mono-N- or di-N,N-(C₁-C₄)alkylamino, cyano, carboxy, (C₁-C₅)alkoxycarbonyl, carbamoyl, formyl or trifluoromethyl and said R₉ rings may optionally be additionally mono- or di-substituted independently with (C₁-C₅)alkyl or halo;

with the proviso that no quaternized nitrogen on any R₉ heterocycle is included;

R₁₂ is morpholino, thiomorpholino, 1-oxothiomorpholino, 1,1-dioxothiomorpholino, thiazolidin-3-yl, 1-oxothiazolidin-3-yl, 1,1-dioxothiazolidin-3-yl, pyrrolidin-1-yl, piperidin-1-yl, piperazin-1-yl, piperazin-4-yl, azetidin-1-yl, 1,2-oxazinan2-yl, pyrazolidin-1-yl, isoxazolidin-2-yl, isothiazolidin-2-yl, 1,2-oxazetidin-2-yl, oxazolidin-3-yl, 3,4-dihydroisoquinolin-2-yl, 1,3-dihydroisoindol-2-yl, 3,4-dihydro-2H-quinol-1-yl, 2,3-dihydro-benzo[1,4]-oxazin-4-yl, 2,3-dihydro-benzo[1,4]-thiazine-4-yl, 3,4-dihydro-2H-quinoxalin-1-yl, 3,4-dihydro-benzo[c][1,2]-oxazin-1-yl, 1,4-dihydro-benzo[d][1,2]-oxazin-3-yl, 3,4-dihydro-benzo[e][1,2]-oxazin-2-yl, 3H-benzo[d]isoxazol-2-yl, 3H-benzo[c]isoxazol-1-yl or azepan-1-yl,

wherein said R₁₂ rings are optionally mono-, di- or tri-substituted independently with halo, (C₁-C₅)alkyl, (C₁-C₅)alkoxy, hydroxy, amino, mono-N- or di-N,N-(C₁-C₅)alkylamino, formyl, carboxy, carbamoyl, mono-N- or di-N,N-(C₁-C₅)alkylcarbamoyl, (C₁-C₆)alkoxy(C₁-C₃)alkoxy, (C₁-C₅)alkoxycarbonyl, benzyloxycarbonyl, (C₁-C₅)alkoxycarbonyl(C₁-C₅)alkyl, (C₁-C₄)alkoxycarbonylamino, carboxy(C₁-C₅)alkyl, carbamoyl(C₁-C₅)alkyl, mono-N- or di-N,N-(C₁-C₅)alkylcarbamoyl(C₁-C₅)alkyl, hydroxy(C₁-C₅)alkyl, (C₁-C₄)alkoxy(C₁-C₄)alkyl, amino(C₁-C₄)alkyl, mono-N- or di-N,N-(C₁-C₄)alkylamino(C₁-C₄)alkyl, oxo, hydroxyimino or (C₁-C₆)alkoxyimino and wherein no more than two substituents are selected from oxo, hydroxyimino or (C₁-C₆)alkoxyimino and oxo, hydroxyimino or (C₁-C₆)alkoxyimino are on nonaromatic carbon; and

wherein said R₁₂ rings are optionally additionally mono- or di-substituted independently with (C₁-C₅)alkyl or halo;

with the proviso that when R₆ is (C₁-C₅)alkoxycarbonyl or benzyloxycarbonyl then R₁ is 5-halo, 5-(C₁-C₄)alkyl or 5-cyano and R₄ is (phenyl)(hydroxy) (C₁-C₄)alkyl, (phenyl)((C₁-C₄)alkoxy) (C₁-C₄)alkyl, hydroxymethyl or Ar(C₁-C₂)alkyl, wherein Ar is thien-2- or -3-yl, fur-2- or -3-yl or phenyl wherein said Ar is optionally mono- or di-substituted independently with halo; with the provisos that when R₄ is benzyl and R₅ is methyl, R₁₂ is not 4-hydroxy-piperidin-1-yl or when R₄ is benzyl and R₅ is methyl R₆ is not C(O)N(CH₃)₂;

with the proviso that when R₁ and R₁₀ and R₁₁ are H, R₄ is not imidazol-4-ylmethyl, 2-phenylethyl or 2-hydroxy-2-phenylethyl;

with the proviso that when R₈ is H and R₉ is (C₁-C₆)alkyl, R₉ is not substituted with carboxy or (C₁-C₄)alkoxycarbonyl on the carbon which is attached to the nitrogen atom N of NHR₉; and

with the proviso that when R₆ is carboxy and R₁, R₁₀, R₁₁, and R₅ are all H, then R₄ is not benzyl, H, (phenyl)(hydroxy)methyl, methyl, ethyl or n-propyl.

The term “compound” as used herein is intended to be interpreted broadly and encompasses organic and inorganic molecules. Organic compounds include, but are not limited to polypeptides, lipids, carbohydrates, coenzymes and nucleic acid molecules.

Polypeptides include but are not limited to antibodies (described in more detail below) and enzymes. Nucleic acids include but are not limited to DNA, RNA and DNA-RNA chimeric molecules. Suitable RNA molecules include RNAi, antisense RNA molecules and ribozymes (all of which are described in more detail below). The nucleic acid can further encode any polypeptide such that administration of the nucleic acid and production of the polypeptide results in an enhancement of glycogen synthesis without substantially impairing responsiveness to glycogenolytic signals.

Administration of nucleic acids that encode a carboxyl terminal deleted G_{M} to a subject for therapeutic purposes is described in more detail in Sections V and VI.

The compound can further be a compound that is identified by any of the screening methods described herein (see the preceding Section).

The compounds of the present invention can optionally be administered in conjunction with other therapeutic agents useful in the treatment of diabetes, other glucose intolerant conditions, hyperphagia or obesity. For example, the compounds of the invention can be administered in conjunction with insulin therapy, hypoglycemic agents, and/or appetite suppressants.

Additional therapeutic agents can optionally be administered concurrently with the compounds of the invention. As used herein, the word “concurrently” means sufficiently close in time to produce a combined effect (that is, concurrently can be simultaneously, or it can be two or more events occurring within a short time period before or after each other).

The compounds of the invention can be a nucleic acid molecule such as an antisense nucleotide sequence or RNAi. The term “antisense nucleotide sequence,” as used herein, refers to a nucleotide sequence that is complementary to a specified DNA or RNA sequence. Antisense RNA sequences and nucleic acids that express the same can be made in accordance with conventional techniques. See, e.g., U.S. Pat. No. 5,023,243 to Tullis; U.S. Pat. No. 5,149,797 to Pederson et al.

Those skilled in the art will appreciate that it is not necessary that the antisense nucleotide sequence be fully complementary to the target sequence as long as the degree of sequence similarity is sufficient for the antisense nucleotide sequence to hybridize to its target and reduce production of the encoded polypeptide (e.g., by at least about 40%, 50%, 60%, 70%, 80%, 90%, 95% or more). As is known in the art, a higher degree of sequence similarity is generally required for short antisense nucleotide sequences, whereas a greater degree of mismatched bases will be tolerated by longer antisense nucleotide sequences.

For example, hybridization of such nucleotide sequences can be carried out under conditions of reduced stringency, medium stringency or even stringent conditions (e.g., conditions represented by a wash stringency of 35-40% Formamide with 5× Denhardt's solution, 0.5% SDS and 1×SSPE at 37° C.; conditions represented by a wash stringency of 40-45% Formamide with 5× Denhardt's solution, 0.5% SDS, and 1×SSPE at 42° C.; and/or conditions represented by a wash stringency of 50% Formamide with 5× Denhardt's solution, 0.5% SDS and 1×SSPE at 42° C., respectively) to the nucleotide sequences specifically disclosed herein. See, e.g., Sambrook et al., Molecular Cloning, A Laboratory Manual (2d Ed. 1989) (Cold Spring Harbor Laboratory).

The antisense nucleotide sequence can be directed against any coding sequence, the silencing of which results in a modulation of an enhancement of glycogen synthesis without substantially impairing responsiveness to glycogenolytic signals.

The length of the antisense nucleotide sequence (i.e., the number of nucleotides therein) is not critical as long as it binds selectively to the intended location and reduces transcription and/or translation of the target sequence (e.g., by at least about 40%, 50%, 60%, 70%, 80%, 90%, 95% or more), and can be determined in accordance with routine procedures. In general, the antisense nucleotide sequence will be from about eight, ten or twelve nucleotides in length up to about 20, 30, or 50 nucleotides, or longer, in length.

An antisense nucleotide sequence can be constructed using chemical synthesis and enzymatic ligation reactions by procedures known in the art. For example, an antisense nucleotide sequence can be chemically synthesized using naturally occurring nucleotides or various modified nucleotides designed to increase the biological stability of the molecules or to increase the physical stability of the duplex formed between the antisense and sense nucleotide sequences, e.g., phosphorothioate derivatives and acridine substituted nucleotides can be used. Examples of modified nucleotides which can be used to generate the antisense nucleotide sequence include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleotide sequence can be produced using an expression vector into which a nucleic acid has been cloned in an antisense orientation (i.e., RNA transcribed from the inserted nucleic acid will be of an antisense orientation to a target nucleic acid of interest).

The antisense nucleotide sequences further include nucleotide sequences wherein at least one, or all, or the internucleotide bridging phosphate residues are modified phosphates, such as methyl phosphonates, methyl phosphonothioates, phosphoromorpholidates, phosphoropiperazidates and phosphoramidates. For example, every other one of the internucleotide bridging phosphate residues can be modified as described. In another non-limiting example, the antisense nucleotide sequence is a nucleotide sequence in which one, or all, of the nucleotides contain a 2′ loweralkyl moiety (e.g., C₁-C₄, linear or branched, saturated or unsaturated alkyl, such as methyl, ethyl, ethenyl, propyl, 1-propenyl, 2-propenyl, and isopropyl). For example, every other one of the nucleotides can be modified as described. See also, Furdon et al., (1989) Nucleic Acids Res. 17, 9193-9204; Agrawal et al., (1990) Proc. Natl. Acad. Sci. USA 87, 1401-1405; Baker et al., (1990) Nucleic Acids Res. 18, 3537-3543; Sproat et al., (1989) Nucleic Acids Res. 17, 3373-3386; Walder and Walder, (1988) Proc. Natl. Acad. Sci. USA 85, 5011-5015; incorporated by reference herein in their entireties for their teaching of methods of making antisense molecules, including those containing modified nucleotide bases).

RNA interference (RNAi) provides another approach for modulating the activity of a polypeptide of interest. The RNAi can be directed against the coding sequence for any polypeptide, where decreased production of that polypeptide results in an enhancement of glycogen synthesis without substantially impairing responsiveness to glycogenolytic signals.

RNAi is a mechanism of post-transcriptional gene silencing in which double-stranded RNA (dsRNA) corresponding to a coding sequence of interest is introduced into a cell or an organism, resulting in degradation of the corresponding mRNA. The RNAi effect persists for multiple cell divisions before gene expression is regained. RNAi is therefore a powerful method for making targeted knockouts or “knockdowns” at the RNA level. RNAi has proven successful in human cells, including human embryonic kidney and HeLa cells (see, e.g., Elbashir et al., Nature (2001) 411:494-8). In one embodiment, silencing can be induced in mammalian cells by enforcing endogenous expression of RNA hairpins (see Paddison et al., (2002), PNAS USA 99:1443-1448). In another embodiment, transfection of small (21-23 nt) dsRNA specifically inhibits nucleic acid expression (reviewed in Caplen, (2002) Trends in Biotechnology 20:49-51).

The mechanism by which RNAi achieves gene silencing has been reviewed in Sharp et al, (2001) Genes Dev 15: 485-490; and Hammond et al., (2001) Nature Rev Gen 2:110-119).

RNAi technology utilizes standard molecular biology methods. dsRNA corresponding to all or a part of a target coding sequence to be inactivated can be produced by standard methods, e.g., by simultaneous transcription of both strands of a template DNA (corresponding to the target sequence) with T7 RNA polymerase. Kits for production of dsRNA for use in RNAi are available commercially, e.g., from New England Biolabs, Inc. Methods of transfection of dsRNA or plasmids engineered to make dsRNA are routine in the art.

Silencing effects similar to those produced by RNAi have been reported in mammalian cells with transfection of a mRNA-cDNA hybrid construct (Lin et al., (2001) Biochem Biophys Res Commun 281:639-44), providing yet another strategy for silencing a coding sequence of interest.

The compound can further be a ribozyme. Ribozymes are RNA-protein complexes that cleave nucleic acids in a site-specific fashion. Ribozymes have specific catalytic domains that possess endonuclease activity (Kim et al., (1987) Proc. Natl. Acad. Sci. USA 84:8788; Gerlach et al., (1987) Nature 328:802; Forster and Symons, (1987) Cell 49:211). For example, a large number of ribozymes accelerate phosphoester transfer reactions with a high degree of specificity, often cleaving only one of several phosphoesters in an oligonucleotide substrate (Michel and Westhof, (1990) J. Mol. Biol. 216:585; Reinhold-Hurek and Shub, (1992) Nature 357:173). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, (1989) Nature 338:217). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., (1991) Proc. Natl. Acad. Sci. USA 88:10591; Sarver et al., (1990) Science 247:1222; Sioud et al., (1992) J. Mol. Biol. 223:831).

A compound of the invention can further be an antibody or antibody fragment. The antibody or antibody fragment can bind to the G_{M} subunit or to any other polypeptide of interest (e.g., PP-1, glycogen synthase, glycogen phosphorylase), as long as the binding between the antibody or the antibody fragment and the target polypeptide results in an enhancement of glycogen synthesis without substantially impairing responsiveness to glycogenolytic signals.

The term “antibody” or “antibodies” as used herein refers to all types of immunoglobulins, including IgG, IgM, IgA, IgD, and IgE. The antibody can be monoclonal or polyclonal and can be of any species of origin, including (for example) mouse, rat, rabbit, horse, or human, or can be a chimeric antibody. See, e.g., Walker et al., Molec. Immunol. 26, 403-11 (1989). The antibodies can be recombinant monoclonal antibodies produced according to the methods disclosed in U.S. Pat. No. 4,474,893 or U.S. Pat. No. 4,816,567. The antibodies can also be chemically constructed according to the method disclosed in U.S. Pat. No. 4,676,980.

Antibody fragments included within the scope of the present invention include, for example, Fab, F(ab′)₂, and Fc fragments, and the corresponding fragments obtained from antibodies other than IgG. Such fragments can be produced by known techniques. For example, F(ab′)₂ fragments can be produced by pepsin digestion of the antibody molecule, and Fab fragments can be generated by reducing the disulfide bridges of the F(ab′)2 fragments. Alternatively, Fab expression libraries can be constructed to allow rapid and easy identification of monoclonal Fab fragments with the desired specificity (Huse et al., (1989) Science 254, 1275-1281).

Polyclonal antibodies used to carry out the present invention can be produced by immunizing a suitable animal (e.g., rabbit, goat, etc.) with an antigen to which a monoclonal antibody to the target binds, collecting immune serum from the animal, and separating the polyclonal antibodies from the immune serum, in accordance with known procedures.

Monoclonal antibodies used to carry out the present invention can be produced in a hybridoma cell line according to the technique of Kohler and Milstein, (1975) Nature 265, 495-97. For example, a solution containing the appropriate antigen can be injected into a mouse and, after a sufficient time, the mouse sacrificed and spleen cells obtained. The spleen cells are then immortalized by fusing them with myeloma cells or with lymphoma cells, typically in the presence of polyethylene glycol, to produce hybridoma cells. The hybridoma cells are then grown in a suitable medium and the supernatant screened for monoclonal antibodies having the desired specificity. Monoclonal Fab fragments can be produced in E. Coli by recombinant techniques known to those skilled in the art. See, e.g., W. Huse, (1989) Science 246, 1275-81.

Antibodies specific to the target polypeptide can also be obtained by phage display techniques known in the art.

V. Delivery Vectors.

The methods of the present invention provide a means for delivering, and optionally expressing, nucleic acids encoding a carboxyl terminal deleted G_{M} subunit in a broad range of host cells, including both dividing and non-dividing cells in vitro (e.g., for large-scale recombinant protein production or for use in screening assays) or in vivo (e.g., for recombinant large-scale protein production, for creating an animal model for disease, or for therapeutic purposes). In embodiments of the invention, the nucleic acid can be expressed transiently in the target cell or the nucleic acid can be stably incorporated into the target cell, for example, by integration into the genome of the cell or by persistent expression from stably maintained episomes (e.g., derived from Epstein Barr Virus).

As one aspect, the vectors, methods and pharmaceutical formulations of the present invention find use in a method of administering a nucleic acid encoding a carboxyl terminal deleted G_{M} subunit to a subject in need thereof. In this manner, a carboxyl terminal deleted G_{M} subunit can thus be produced in vivo in the subject, where the production of a carboxyl terminal deleted G_{M} subunit in the subject can impart some therapeutic effect. Pharmaceutical formulations and methods of delivering nucleic acids encoding a carboxyl terminal deleted G_{M} subunit for therapeutic purposes are described in more detail in Section VI below.

Alternatively, an isolated nucleic acid encoding a carboxyl terminal deleted G_{M} subunit can be administered to a subject so that the nucleic acid is expressed by the subject and a carboxyl terminal deleted G_{M} subunit is produced and purified therefrom, i.e., as a source of recombinant the carboxyl terminal deleted G_{M} subunit protein. According to this embodiment, it is preferred that the carboxyl terminal deleted G_{M} subunit is secreted into the systemic circulation or into another body fluid (e.g., milk, lymph, spinal fluid, urine) that is easily collected and from which the protein can be further purified. As a further alternative, the carboxyl terminal deleted G_{M} subunit protein can be produced in avian species and deposited in, and conveniently isolated from, egg proteins.

Likewise, nucleic acids encoding a carboxyl terminal deleted G_{M} subunit can be expressed transiently or stably in a cell culture system for the purpose of screening assays (described in Section III above) or for large-scale recombinant protein production. The cell can be a bacterial, protozoan, plant, yeast, fungus, or animal cell. Preferably, the cell is an animal cell (e.g., insect, avian or mammalian), and more preferably a mammalian cell (e.g., a fibroblast). Cells for use in the screening methods of the invention are described in more detail hereinabove.

It will be apparent to those skilled in the art that any suitable vector can be used to deliver the isolated nucleic acids of this invention to the target cell(s) or subject of interest. The choice of delivery vector can be made based on a number of factors known in the art, including age and species of the target host, in vitro vs. in vivo delivery, level and persistence of expression desired, intended purpose (e.g., for therapy or drug screening), the target cell or organ, route of delivery, size of the isolated nucleic acid, safety concerns, and the like.

Suitable vectors include virus vectors (e.g., retrovirus, alphavirus; vaccinia virus; adenovirus, adeno-associated virus, or herpes simplex virus), lipid vectors, poly-lysine vectors, synthetic polyamino polymer vectors that are used with nucleic acid molecules, such as plasmids, and the like.

Any viral vector that is known in the art can be used in the present invention. Examples of such viral vectors include, but are not limited to vectors derived from: Adenoviridae; Birnaviridae; Bunyaviridae; Caliciviridae, Capillovirus group; Carlavirus group; Carmovirus virus group; Group Caulimovirus; Closterovirus Group; Commelina yellow mottle virus group; Comovirus virus group; Coronaviridae; PM2 phage group; Corcicoviridae; Group Cryptic virus; group Cryptovirus; Cucumovirus virus group Family ([PHgr]6 phage group; Cysioviridae; Group Carnation ringspot; Dianthovirus virus group; Group Broad bean wilt; Fabavirus virus group; Filoviridae; Flaviviridae; Furovirus group; Group Germinivirus; Group Giardiavirus; Hepadnaviridae; Herpesviridae; Hordeivirus virus group; Illarvirus virus group; Inoviridae; Iridoviridae; Leviviridae; Lipothrixviridae; Luteovirus group; Marafivirus virus group; Maize chlorotic dwarf virus group; icroviridae; Myoviridae; Necrovirus group; Nepovirus virus group; Nodaviridae; Orthomyxoviridae; Papovaviridae; Paramyxoviridae; Parsnip yellow fleck virus group; Partitiviridae; Parvoviridae; Pea enation mosaic virus group; Phycodnaviridae; Picomaviridae; Plasmaviridae; Prodoviridae; Polydnaviridae; Potexvirus group; Potyvirus; Poxyiridae; Reoviridae; Retroviridae; Rhabdoviridae; Group Rhizidiovirus; Siphoviridae; Sobemovirus group; SSV 1-Type Phages; Tectiviridae; Tenuivirus; Tetraviridae; Group Tobamovirus; Group Tobravirus; Togaviridae; Group Tombusvirus; Group Torovirus; Totiviridae; Group Tymovirus; and Plant virus satellites.

Protocols for producing recombinant viral vectors and for using viral vectors for nucleic acid delivery can be found in Current Protocols in Molecular Biology, Ausubel, F. M. et al. (eds.) Greene Publishing Associates, (1989) and other standard laboratory manuals (e.g., Vectors for Gene Therapy. In: Current Protocols in Human Genetics. John Wiley and Sons, Inc.: 1997). Particular examples of viral vectors are those previously employed for the delivery of nucleic acids including, for example, retrovirus, adenovirus, AAV, herpes virus, and poxvirus vectors.

In certain embodiments of the present invention, the delivery vector is an adenovirus vector. The term “adenovirus” as used herein is intended to encompass all adenoviruses, including the Mastadenovirus and Aviadenovirus genera. To date, at least forty-seven human serotypes of adenoviruses have been identified (see, e.g., FIELDS et al., VIROLOGY, volume 2, chapter 67 (3d ed., Lippincott-Raven Publishers). Preferably, the adenovirus is a serogroup C adenovirus, still more preferably the adenovirus is serotype 2 (Ad2) or serotype 5 (Ad5).

The various regions of the adenovirus genome have been mapped and are understood by those skilled in the art (see, e.g., FIELDS et al., VIROLOGY, volume 2, chapters 67 and 68 (3d ed., Lippincott-Raven Publishers). The genomic sequences of the various Ad serotypes, as well as the nucleotide sequence of the particular coding regions of the Ad genome, are known in the art and can be accessed, e.g., from GenBank and NCBI (see, e.g., GenBank Accession Nos. J0917, M73260, X73487, AF108105, L19443, NC 003266 and NCBI Accession Nos. NC 001405, NC 001460, NC 002067, NC 00454).

Those skilled in the art will appreciate that the inventive adenovirus vectors can be modified or “targeted” as described in Douglas et al., (1996) Nature Biotechnology 14:1574; U.S. Pat. No. 5,922,315 to Roy et al.; U.S. Pat. No. 5,770,442 to Wickham-et al.; and/or U.S. Pat. No. 5,712,136 to Wickham et al.

An adenovirus vector genome or rAd vector genome will typically comprise the Ad terminal repeat sequences and packaging signal. An “adenovirus particle” or “recombinant adenovirus particle” comprises an adenovirus vector genome or recombinant adenovirus vector genome, respectively, packaged within an adenovirus capsid. Generally, the adenovirus vector genome is most stable at sizes of about 28 kb to 38 kb (approximately 75% to 105% of the native genome size). In the case of an adenovirus vector containing large deletions and a relatively small heterologous nucleic acid of interest, “stuffer DNA” can be used to maintain the total size of the vector within the desired range by methods known in the art.

Normally adenoviruses bind to a cell surface receptor (CAR) of susceptible cells via the knob domain of the fiber protein on the virus surface. The fiber knob receptor is a 45 kDa cell surface protein which has potential sites for both glycosylation and phosphorylation. (Bergelson et al., (1997), Science 275:1320-1323). A secondary method of entry for adenovirus is through integrins present on the cell surface. Arginine-Glycine-Aspartic Acid (RGD) sequences of the adenoviral penton base protein bind integrins on the cell surface.

The adenovirus genome can be manipulated such that it encodes and expresses a nucleic acid of interest but is inactivated in terms of its ability to replicate in a normal lytic viral life cycle. See, for example, Berkner et al. (1988) BioTechniques 6:616; Rosenfeld et al. (1991) Science 252:431-434; and Rosenfeld et al. (1992) Cell 68:143-155. Representative adenoviral vectors derived from the adenovirus strain Ad type 5 d1324 or other strains of adenovirus (e.g., Ad2, Ad3, Ad7 etc.) are known to those skilled in the art.

Recombinant adenoviruses can be advantageous in certain circumstances in that they are not capable of infecting nondividing cells and can be used to infect a wide variety of cell types, including epithelial cells. Furthermore, the virus particle is relatively stable and amenable to purification and concentration, and can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., as occurs with retroviral DNA). Moreover, the carrying capacity of the adenoviral genome for foreign DNA is large relative to other delivery vectors (Haj-Ahmand and Graham (1986) J. Virol. 57:267).

In particular embodiments, the adenovirus genome contains a deletion therein, so that at least one of the adenovirus genomic regions does not encode a functional protein. For example, first-generation adenovirus vectors are typically deleted for the E1 genes and packaged using a cell that expresses the E1 proteins (e.g., 293 cells). The E3 region is also frequently deleted as well, as there is no need for complementation of this deletion. In addition, deletions in the E4, E2a, protein IX, and fiber protein regions have been described, e.g., by Armentano et al, (1997) J. Virology 71:2408, Gao et al., (1996) J. Virology 70:8934, Dedieu et al., (1997) J. Virology 71;4626, Wang et al., (1997) Gene Therapy 4:393, U.S. Pat. No. 5,882,877 to Gregory et al. (the disclosures of which are incorporated herein in their entirety). Preferably, the deletions are selected to avoid toxicity to the packaging cell. Wang et al., (1997) Gene Therapy 4:393, has described toxicity from constitutive co-expression of the E4 and E1 genes by a packaging cell line. Toxicity can be avoided by regulating expression of the E1 and/or E4 gene products by an inducible, rather than a constitutive, promoter. Combinations of deletions that avoid toxicity or other deleterious effects on the host cell can be routinely selected by those skilled in the art.

As further examples, in particular embodiments, the adenovirus is deleted in the polymerase (pol), preterminal protein (pTP), IVa2 and/or 100K regions (see, e.g., U.S. Pat. No. 6,328,958; PCT publication WO 00/12740; and PCT publication WO 02/098466; Ding et al., (2002) Mol. Ther. 5:436; Hodges et al., J. Virol. 75:5913; Ding et al., (2001) Hum Gene Ther 12:955; the disclosures of which are incorporated herein by reference in their entireties for the teachings of how to make and use deleted adenovirus vectors for gene delivery).

The term “deleted” adenovirus as used herein refers to the omission of at least one nucleotide from the indicated region of the adenovirus genome. Deletions can be greater than about 1, 2, 3, 5, 10, 20, 50, 100, 200, or even 500 nucleotides. Deletions in the various regions of the adenovirus genome can be about at least 1%, 5%, 10%, 25%, 50%, 75%, 90%, 95%, 99%, or more of the indicated region. Alternately, the entire region of the adenovirus genome is deleted. Preferably, the deletion will prevent or essentially prevent the expression of a functional protein from that region. In general, larger deletions are preferred as these have the additional advantage that they will increase the carrying capacity of the deleted adenovirus for a heterologous nucleotide sequence of interest. The various regions of the adenovirus genome have been mapped and are understood by those skilled in the art (see, e.g., FIELDS et al., VIROLOGY, volume 2, chapters 67 and 68 (3d ed., Lippincott-Raven Publishers).

Those skilled in the art will appreciate that typically, with the exception of the E3 genes, any deletions will need to be complemented in order to propagate (replicate and package) additional virus, e.g., by transcomplementation with a packaging cell.

The present invention can also be practiced with “gutted” adenovirus vectors (as that term is understood in the art, see e.g., Lieber et al., (1996) J. Virol. 70:8944-60) in which essentially all of the adenovirus genomic sequences are deleted.

Adeno-associated viruses (AAV) have also been employed as nucleic acid delivery vectors. For a review, see Muzyczka et al. Curr. Topics in Micro. and Immunol. (1992) 158:97-129). MV are parvoviruses and have small icosahedral virions, 18-26 nanometers in diameter and contain a single stranded genomic DNA molecule 4-5 kilobases in size. The viruses contain either the sense or antisense strand of the DNA molecule and either strand is incorporated into the virion. Two open reading frames encode a series of Rep and Cap polypeptides. Rep polypeptides (Rep50, Rep52, Rep68 and Rep78) are involved in replication, rescue and integration of the MV genome, although significant activity can be observed in the absence of all four Rep polypeptides. The Cap proteins (VP1, VP2, VP3) form the virion capsid. Flanking the rep and cap open reading frames at the 5′ and 3′ ends of the genome are 145 basepair inverted terminal repeats (ITRs), the first 125 basepairs of which are capable of forming Y- or T-shaped duplex structures. It has been shown that the ITRs represent the minimal cis sequences required for replication, rescue, packaging and integration of the MV genome. Typically, in recombinant AAV vectors (rAAV), the entire rep and cap coding regions are excised and replaced with a heterologous nucleic acid of interest.

AAV are among the few viruses that can integrate their DNA into non-dividing cells, and exhibit a high frequency of stable integration into human chromosome 19 (see, for example, Flotte et al. (1992) Am. J. Respir. Cell. Mol. Biol. 7:349-356; Samulski et al., (1989) J. Virol. 63:3822-3828; and McLaughlin et al., (1989) J. Virol. 62:1963-1973). A variety of nucleic acids have been introduced into different cell types using AAV vectors (see, for example, Hermonat et al., (1984) Proc. Natl. Acad. Sci. USA 81:6466-6470; Tratschin et al., (1985) Mol. Cell. Biol. 4:2072-2081; Wondisford et al., (1988) Mol. Endocrinol. 2:32-39; Tratschin et al., (1984) J. Virol. 51:611-619; and Flotte et al., (1993) J. Biol. Chem. 268:3781-3790).

A rAAV vector genome will typically comprise the MV terminal repeat sequences and packaging signal. An “MV particle” or “rAAV particle” comprises an AAV vector genome or rAAV vector genome, respectively, packaged within an AAV capsid. The rMV vector itself need not contain AAV genes encoding the capsid and Rep proteins. In particular embodiments of the invention, the rep and/or cap genes are deleted from the MV genome. In a representative embodiment, the rAAV vector retains only the terminal AAV sequences (ITRs) necessary for integration, excision, replication.

Sources for the MV capsid genes can include serotypes MV-1, AAV-2, AAV-3 (including 3a and 3b), MV-4, AAV-5, MV-6, MV-7, AAV-8, as well as bovine MV and avian MV, and any other virus classified by the International Committee on Taxonomy of Viruses (ICTV) as an MV (see, e.g., BERNARD N. FIELDS et al., VIROLOGY, volume 2, chapter 69 (4th ed., Lippincott-Raven Publishers).

Because of packaging limitations, the total size of the rMV genome will preferably be less than about 5.2, 5, 4.8, 4.6 or 4.5 kb in size.

Any suitable method known in the art can be used to produce AAV vectors expressing the nucleic acids of this invention, for example, coding sequences for a carboxyl terminal deleted G_{M} subunit (see, e.g., U.S. Pat. No. 5,139,941; U.S. Pat. No. 5,858,775; U.S. Pat. No. 6,146,874 for illustrative methods). In one particular method, AAV stocks can be produced by co-transfection of a rep/cap vector encoding MV packaging functions and the template encoding the MV vDNA into human cells infected with the helper adenovirus (Samulski et al., (1989) J. Virology 63:3822).

In other particular embodiments, the adenovirus helper virus is a hybrid helper virus that encodes AAV Rep and/or capsid proteins. Hybrid helper Ad/AAV vectors expressing AAV rep and/or cap genes and methods of producing AAV stocks using these reagents are known in the art (see, e.g., U.S. Pat. No. 5,589,377; and U.S. Pat. No. 5,871,982, U.S. Pat. No. 6,251,677; and U.S. Pat. No. 6,387,368). Preferably, the hybrid Ad of the invention expresses the AAV capsid proteins (i.e., VP1, VP2, and VP3). Alternatively, or additionally, the hybrid adenovirus can express one or more of AAV Rep proteins (i.e., Rep40, Rep52, Rep68 and/or Rep78). The AAV sequences can be operatively associated with a tissue-specific or inducible promoter.

The AAV rep and/or cap genes can alternatively be provided by a packaging cell that stably expresses the genes (see, e.g., Gao et al., (1998) Human Gene Therapy 9:2353; Inoue et al., (1998) J. Virol. 72:7024; U.S. Pat. No. 5,837,484; WO 98/27207; U.S. Pat. No. 5,658,785; WO 96/17947).

Another vector for use in the present invention comprises Herpes Simplex Virus (HSV). Herpes simplex virions have an overall diameter of 150 to 200 nm and a genome consisting of one double-stranded DNA molecule that is 120 to 200 kilobases in length. Glycoprotein D (gD) is a structural component of the HSV envelope that mediates virus entry into host cells. The initial interaction of HSV with cell surface heparin sulfate proteoglycans is mediated by another glycoprotein, glycoprotein C (gC) and/or glycoprotein B (gB). This is followed by interaction with one or more of the viral glycoproteins with cellular receptors. It has been shown that glycoprotein D of HSV binds directly to Herpes virus entry mediator (HVEM) of host cells. HVEM is a member of the tumor necrosis factor receptor superfamily (Whitbeck et al., (1997), J. Virol; 71:6083-6093). Finally, gD, gB and the complex of gH and gL act individually or in combination to trigger pH-independent fusion of the viral envelope with the host cell plasma membrane. The virus itself is transmitted by direct contact and replicates in the skin or mucosal membranes before infecting cells of the nervous system for which HSV has particular tropism. It exhibits both a lytic and a latent function. The lytic cycle results in viral replication and cell death. The latent function allows for the virus to be maintained in the host for an extremely long period of time.

HSV can be modified for the delivery of nucleic acids to cells by producing a vector that exhibits only the latent function for long-term gene maintenance. HSV vectors are useful for nucleic acid delivery because they allow for a large DNA insert of up to or greater than 20 kilobases; they can be produced with extremely high titers; and they have been shown to express nucleic acids for a long period of time in the central nervous system as long as the lytic cycle does not occur.

In other particular embodiments of the present invention, the delivery vector of interest is a retrovirus. Retroviruses normally bind to a virus-specific cell surface receptor, e.g., CD4 (for HIV); CAT (for MLV-E; ecotropic Murine leukemic virus E); RAM1/GLVR2 (for murine leukemic virus-A; MLV-A); GLVR1 (for Gibbon Ape leukemia virus (GALV) and Feline leukemia virus B (FeLV-B)). The development of specialized cell lines (termed “packaging cells”) which produce only replication-defective retroviruses has increased the utility of retroviruses for gene therapy, and defective retroviruses are characterized for use in gene transfer for gene therapy purposes (for a review, see Miller, (1990) Blood 76:271). A replication-defective retrovirus can be packaged into virions which can be used to infect a target cell through the use of a helper virus by standard techniques.

Yet another suitable vector is a poxvirus vector. These viruses are very complex, containing more than 100 proteins, although the detailed structure of the virus is presently unknown. Extracellular forms of the virus have two membranes while intracellular particles only have an inner membrane. The outer surface of the virus is made up of lipids and proteins that surround the biconcave core. Poxviruses are antigenically complex, inducing both specific and cross-reacting antibodies after infection. Poxvirus receptors are not presently known, but it is likely that there exists more than one given the tropism of poxvirus for a wide range of cells. Poxvirus gene expression is well studied due to the interest in using vaccinia virus as a vector for expression of nucleic acids.

In addition to viral transfer methods, such as those illustrated above, non-viral methods can also be employed. Many non-viral methods of nucleic acid transfer rely on normal mechanisms used by mammalian cells for the uptake and intracellular transport of macromolecules. In particular embodiments, non-viral nucleic acid delivery systems rely on endocytic pathways for the uptake of the nucleic acid molecule by the targeted cell. Exemplary nucleic acid delivery systems of this type include liposomal derived systems, poly-lysine conjugates, and artificial viral envelopes.

In particular embodiments, plasmid vectors are used in the practice of the present invention. Naked plasmids can be introduced into muscle cells by injection into the tissue. Expression can extend over many months, although the number of positive cells is typically low (Wolff et al., (1989) Science 247:247). Cationic lipids have been demonstrated to aid in introduction of nucleic acids into some cells in culture (Felgner and Ringold, (1989) Nature 337:387). Injection of cationic lipid plasmid DNA complexes into the circulation of mice has been shown to result in expression of the DNA in lung (Brigham et al., (1989) Am. J. Med. Sci. 298:278). One advantage of plasmid DNA is that it can be introduced into non-replicating cells.

In a representative embodiment, a nucleic acid molecule (e.g., a plasmid) can be entrapped in a lipid particle bearing positive charges on its surface and, optionally, tagged with antibodies against cell surface antigens of the target tissue (Mizuno et al., (1992) No Shinkei Geka 20:547; PCT publication WO 91/06309; Japanese patent application 1047381; and European patent publication EP-A-43075).

Liposomes that consist of amphiphilic cationic molecules are useful non-viral vectors for nucleic acid delivery in vitro and in vivo (reviewed in Crystal, Science 270: 404-410 (1995); Blaese et al., Cancer Gene Ther. 2: 291-297 (1995); Behr et al., Bioconjugate Chem. 5: 382-389 (1994); Remy et al., Bioconjugate Chem. 5: 647-654 (1994); and Gao et al., Gene Therapy 2: 710-722 (1995)). The positively charged liposomes are believed to complex with negatively charged nucleic acids via electrostatic interactions to form lipid:nucleic acid complexes. The lipid:nucleic acid complexes have several advantages as nucleic acid transfer vectors. Unlike viral vectors, the lipid:nucleic acid complexes can be used to transfer expression cassettes of essentially unlimited size. Since the complexes lack proteins, they can evoke fewer immunogenic and inflammatory responses. Moreover, they cannot replicate or recombine to form an infectious agent and have low integration frequency. A number of publications have demonstrated that amphiphilic cationic lipids can mediate nucleic acid delivery in vivo and in vitro (Felgner et al., Proc. Natl. Acad. Sci. USA 84: 7413-17 (1987); Loeffler et al., Methods in Enzymology 217: 599-618 (1993); Felgner et al., J. Biol. Chem. 269: 2550-2561 (1994)).

Several groups have reported the use of amphiphilic cationic lipid:nucleic acid complexes for in vivo transfection both in animals and in humans (reviewed in Gao et al., Gene Therapy 2: 710-722 (1995); Zhu et al., Science 261: 209-211 (1993); and Thierry et al., Proc. Natl. Acad. Sci. USA 92: 9742-9746 (1995)). U.S. Pat. No. 6,410,049 describes a method of preparing cationic lipid:nucleic acid complexes that have a prolonged shelf life.

VI. Subjects, Pharmaceutical Formulations, Dosages and Modes of Administration.

The present invention finds use in veterinary and medical applications. Suitable subjects include both avians and mammals, with mammals being preferred. The term “avian” as used herein includes, but is not limited to, chickens, ducks, geese, quail, turkeys and pheasants. The term “mammal” as used herein includes, but is not limited to, humans, bovines, ovines, caprines, equines, felines, canines, lagomorphs, etc. Human subjects include neonates, infants, juveniles, and adults.

In particular embodiments, the subject has impaired glucose tolerance, has been diagnosed with diabetes mellitus (e.g., non-insulin dependent, insulin dependent, MODY-2), has acute or chronic hyperglycemia, is obese, and/or has hyperphagia. In other particular embodiments, the subject has low hepatic glucokinase activity (e.g., a subject with MODY-2). In still other embodiments, the subject is a mammal, for example, an animal model for diabetes mellitus. In yet other embodiments, the subject is a mammalian subject that excretes glucose in its urine.

As one particular aspect, the invention provides a pharmaceutical formulation comprising a compound that enhances glycogen synthesis but does not substantially impair responsiveness to glycogenolytic signals in a pharmaceutically acceptable carrier, e.g., as a method of reducing caloric intake or reducing hyperglycemia. In representative embodiments, the compound modulates the activity of a G_{M} and/or carboxyl terminal deleted G_{M} subunit. As another aspect, the present invention provides a pharmaceutical formulation comprising a compound identified according to the screening methods of this invention in a pharmaceutically acceptable carrier.

In other particular embodiments, the present invention provides a pharmaceutical composition comprising a delivery vector of the invention in a pharmaceutically-acceptable carrier.

By “pharmaceutically acceptable” it is meant a material that is not biologically or otherwise undesirable, i.e., the material can be administered to a subject without causing any undesirable biological effects such as toxicity.

The formulations of the invention can optionally comprise medicinal agents, pharmaceutical agents, carriers, adjuvants, dispersing agents, diluents, and the like.

The compounds of the invention can be formulated for administration in a pharmaceutical carrier in accordance with known techniques. See, e.g., Remington, The Science And Practice of Pharmacy (9^{th} Ed. 1995). In the manufacture of a pharmaceutical formulation according to the invention, the compound (including the physiologically acceptable salts thereof) is typically admixed with, inter alia, an acceptable carrier. The carrier can be a solid or a liquid, or both, and is preferably formulated with the compound as a unit-dose formulation, for example, a tablet, which can contain from 0.01 or 0.5% to 95% or 99% by weight of the compound. One or more compounds can be incorporated in the formulations of the invention, which can be prepared by any of the well-known techniques of pharmacy.

A further aspect of the invention is a method of treating subjects in vivo with the inventive compounds of the invention, comprising administering to a subject a pharmaceutical composition comprising a compound of the invention in a pharmaceutically acceptable carrier, wherein the pharmaceutical composition is administered in a therapeutically effective amount. Administration of the compounds of the present invention to a human subject or an animal in need thereof can be by any means known in the art for administering compounds.

One particular embodiment of the invention is a method of reducing caloric intake comprising administering a compound of the invention to a subject in an amount effective to reduce caloric intake, where the compound is a glycogen synthesis enhancer but does not substantially impair responsiveness to glycogenolytic signals. According to another embodiment, the invention provides a method of reducing hyperglycemia by administering to a subject a compound of the invention in an amount to reduce hyperglycemia, where the compound is a glycogen synthesis enhancer but does not substantially impair responsiveness to glycogenolytic signals.

In one particular embodiment, the invention provides a method of reducing caloric intake by administering to a subject a pharmaceutical composition comprising a glycogen phosphorylase inhibitor in a pharmaceutically acceptable carrier in an amount effective to reduce caloric intake in the subject. Such compounds include, without limitation, substituted n-(indole-2-carbonyl-) amides, substituted n-(indole-2-carbonyl-) glycinamides, and derivatives of the foregoing (described above).

The formulations of the invention include those suitable for oral, rectal, topical, buccal (e.g., sub-lingual), vaginal, parenteral (e.g., subcutaneous, intramuscular including skeletal muscle, cardiac muscle, diaphragm muscle and smooth muscle, intradermal, intravenous, intraperitoneal), topical (i.e., both skin and mucosal surfaces, including airway surfaces), intranasal, transdermal, intraarticular, intrathecal and inhalation administration, administration to the liver, as well as direct organ injection (e.g., into the liver, into the brain for delivery to the central nervous system, into the pancreas). The most suitable route in any given case will depend on the nature and severity of the condition being treated and on the nature of the particular compound which is being administered.

In particularly preferred embodiments of the invention, the compound is delivered to the liver of the subject. Administration to the liver-can be achieved by any method known in the art, including, but not limited to intravenous administration, intraportal administration, intrabiliary administration, intra-arterial administration, and direct injection into the liver parenchyma.

Delivery to skeletal muscle may be by any method known in the art, including intravenous administration, intramuscular injection, topical, intradermal or subcutaneous injection in proximity to skeletal muscle, or implantation of a slow release depot comprising the compound.

For injection, the carrier will typically be a liquid, such as sterile pyrogen-free water, pyrogen-free phosphate-buffered saline solution, bacteriostatic water, or Cremophor EL[R] (BASF, Parsippany, N.J.). For other methods of administration, the carrier can be either solid or liquid.

For oral administration, the compound can be administered in solid dosage forms, such as capsules, tablets, and powders, or in liquid dosage forms, such as elixirs, syrups, and suspensions. Compounds can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate and the like. Examples of additional inactive ingredients that can be added to provide desirable color, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for buccal (sub-lingual) administration include lozenges comprising the compound in a flavored base, usually sucrose and acacia or tragacanth; and pastilles comprising the compound in an inert base such as gelatin and glycerin or sucrose and acacia.

Formulations of the present invention suitable for parenteral administration comprise sterile aqueous and non-aqueous injection solutions of the compound, which preparations are preferably isotonic with the blood of the intended recipient. These preparations can contain anti-oxidants, buffers, bacteriostats and solutes which render the formulation isotonic with the blood of the intended recipient. Aqueous and non-aqueous sterile suspensions can include suspending agents and thickening agents. The formulations can be presented in unit\dose or multi-dose containers, for example sealed ampoules and vials, and can be stored in a freeze-dried (lyophilized) condition requiring only the addition of the sterile liquid carrier, for example, saline or water-for-injection immediately prior to use.

Extemporaneous injection solutions and suspensions can be prepared from sterile powders, granules and tablets of the kind previously described. For example, in one aspect of the present invention, there is provided an injectable, stable, sterile composition comprising a compound of the invention, in a unit dosage form in a sealed container. The compound or salt is provided in the form of a lyophilizate which is capable of being reconstituted with a suitable pharmaceutically acceptable carrier to form a liquid composition suitable for injection thereof into a subject. The unit dosage form typically comprises from about 10 mg to about 10 grams of the compound or salt. When the compound or salt is substantially water-insoluble, a sufficient amount of emulsifying agent which is pharmaceutically acceptable can be employed in sufficient quantity to emulsify the compound or salt in an aqueous carrier. One such useful emulsifying agent is phosphatidyl choline.

Formulations suitable for rectal administration are preferably presented as unit dose suppositories. These can be prepared by admixing the compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which can be used include petroleum jelly, lanoline, polyethylene glycols, alcohols, transdermal enhancers, and combinations of two or more thereof.

Further, the administration may be acute or chronic as needed. For example, acute administration can be suitable for short-term or episodic treatment, whereas chronic administration can be more desirable for long-term treatment.

Formulations suitable for transdermal administration can be presented as discrete patches adapted to remain in intimate contact with the epidermis of the recipient for a prolonged period of time. Formulations suitable for transdermal administration can also be delivered by iontophoresis (see, for example, Pharmaceutical Research 3 (6):318 (1986)) and typically take the form of an optionally buffered aqueous solution of the compound. Suitable formulations comprise citrate or bis\tris buffer (pH 6) or ethanol/water and contain from 0.1 to 0.2M of the compound.

The compound can alternatively be formulated for nasal administration or otherwise administered to the lungs of a subject by any suitable means, but is preferably administered by an aerosol suspension of respirable particles comprising the compound, which the subject inhales. The respirable particles can be liquid or solid. The term “aerosol” includes any gas-borne suspended phase, which is capable of being inhaled into the bronchioles or nasal passages. Specifically, aerosol includes a gas-borne suspension of droplets, as can be produced in a metered dose inhaler or nebulizer, or in a mist sprayer. Aerosol also includes a dry powder composition suspended in air or other carrier gas, which can be delivered by insufflation from an inhaler device, for example. See Ganderton & Jones, Drug Delivery to the Respiratory Tract, Ellis Horwood (1987); Gonda (1990) Critical Reviews in Therapeutic Drug Carrier Systems 6:273-313; and Raeburn et al. (1992) J. Pharmacol. Toxicol. Methods 27:143-159. Aerosols of liquid particles comprising the compound can be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the compound can likewise be produced with any solid particulate medicament aerosol generator, by techniques known in the pharmaceutical art.

Alternatively, one can administer the compound in a local rather than systemic manner, for example, in a depot or sustained-release formulation.

Further, the present invention provides liposomal formulations of the compounds disclosed herein and salts thereof. The technology for forming liposomal suspensions is well known in the art. When the compound or salt thereof is an aqueous-soluble salt, using conventional liposome technology, the same can be incorporated into lipid vesicles. In such an instance, due to the water solubility of the compound or salt, the compound or salt will be substantially entrained within the hydrophilic center or core of the liposomes. The lipid layer employed can be of any conventional composition and can either contain cholesterol or can be cholesterol-free. When the compound or salt of interest is water-insoluble, again employing conventional liposome formation technology, the salt can be substantially entrained within the hydrophobic lipid bilayer which forms the structure of the liposome. In either instance, the liposomes which are produced can be reduced in size, as through the use of standard sonication and homogenization techniques.

The liposomal formulations containing the compounds disclosed herein or salts thereof, can be lyophilized to produce a lyophilizate which can be reconstituted with a pharmaceutically acceptable carrier, such as water, to regenerate a liposomal suspension.

In the case of water-insoluble compounds, a pharmaceutical composition can be prepared containing the water-insoluble compound, such as for example, in an aqueous base emulsion. In such an instance, the composition will contain a sufficient amount of pharmaceutically acceptable emulsifying agent to emulsify the desired amount of the compound. Particularly useful emulsifying agents include phosphatidyl cholines and lecithin.

In particular embodiments, the compound is administered to the subject in a therapeutically effective amount, as that term is defined above. Dosages of pharmaceutically active compounds can be determined by methods known in the art, see, e.g., Remington's Pharmaceutical Sciences (Maack Publishing Co., Easton, Pa.) The therapeutically effective dosage of any specific compound, the use of which is in the scope of present invention, will vary somewhat from compound to compound, and patient to patient, and will depend upon the condition of the patient and the route of delivery. As a general proposition, a dosage from about 0.1 to about 50 mg/kg will have therapeutic efficacy, with all weights being calculated based upon the weight of the compound, including the cases where a salt is employed. Toxicity concerns at the higher level can restrict intravenous dosages to a lower level such as up to about 10 mg/kg, with all weights being calculated based upon the weight of the compound, including the cases where a salt is employed. A dosage from about 10 mg/kg to about 50 mg/kg can be employed for oral administration. Typically, a dosage from about 0.5 mg/kg to 5 mg/kg can be employed for intramuscular injection. Particular dosages are about 1 μmol/kg to 50 μmol/kg, and more particularly about 22 μmol/kg and 33 μmol/kg of the compound for intravenous or oral administration.

A further aspect of the invention is a method of treating subjects in vivo with the inventive delivery vectors of this invention. Administration of the delivery vectors of the present invention to a human or an animal subject can be by any means known in the art for administering vectors. Subjects are as described hereinabove.

Accordingly, in a further embodiment, the present invention provides a method of reducing caloric intake in a subject (e.g., with diabetes mellitus) comprising administering to the subject an isolated nucleic acid comprising a nucleotide sequence encoding a carboxyl terminal deleted G_{M} subunit in an amount effective to reduce caloric intake. In alternative embodiments, the invention provides a method of decreasing hyperphagia in a subject comprising administering to the subject an isolated nucleic acid comprising a nucleotide sequence encoding a carboxyl terminal deleted G_{M} subunit in an amount effective to reduce hyperphagia.

As still a further aspect, the invention encompasses a method of reducing hyperglycemia in a subject in need thereof (e.g., a subject with diabetes mellitus) comprising administering to the subject an isolated nucleic acid comprising a nucleotide sequence encoding a carboxyl terminal deleted G_{M} subunit in an amount effective to reduce hyperglycemia.

The treatments of the invention may administered in combination with any other medical regimen known in the art, e.g, in combination with insulin, oral hyperglycemic agents, or appetite suppressants.

Dosages of the delivery vectors will depend upon the mode of administration, the severity of the disease or condition to be treated, the individual subject's condition, age and species of the subject, the particular vector, and the nucleic acid to be delivered, and can be determined in a routine manner. In particular embodiments, the vector is administered to the subject in a therapeutically effective amount, as that term is defined above.

Typically, with respect to viral vectors, at least about 10³ virus particles, at least about 10⁵ virus particles, at least about 10⁷ virus particles, at least about 10⁹ virus particles, at least about 10¹¹ virus particles, at least about 10¹² virus particles, or at least about 10¹³ virus particles are administered to the subject per treatment. Exemplary doses are virus titers of about 10⁷ to about 10¹⁵ particles, about 10⁷ to about 10¹⁴ particles, about 10⁸ to about 10¹³ particles, about 10¹⁰ to about 10¹⁵ particles, about 10¹¹ to about 10¹⁵ particles, about 10¹² to about 10¹⁴ particles, or about 10¹² to about 10¹³ particles.

In particular embodiments of the invention, more than one administration (e.g., two, three, four, or more administrations) can be employed over a variety of time intervals (e.g., hourly, daily, weekly, monthly, etc.) to achieve-therapeutic levels of nucleic acid expression.

Having now described the invention, the same will be illustrated with reference to certain examples, which are included herein for illustration purposes only, and which are not intended to be limiting of the invention.

### EXAMPLE 1

### Recombinant Adenoviruses

A recombinant adenovirus containing the cDNA that encodes for a truncated G_{M} glycogen targeting subunit isoform from which 735 C-terminal amino acids were removed (G_{M}ΔC) is termed AdCMV-G_{M}ΔC; its preparation has been described previously (Yang et al. (2002) J. Biol. Chem. 277:1514-1523). The coding sequence for G_{M}ΔC is shown in FIG. 8 (SEQ ID NO:9) and the amino acid sequence of the truncated subunit is shown in SEQ ID NO:10. The cDNA insert includes a C-terminal FLAG tag for ready identification of the transgene product by immunoblotting (Yang et al. (2002) J Biol. Chem. 277:1514-1523). As a control, some animals received a virus containing the E. coli β-galactosidase gene, termed AdCMV-βGAL (Herz and Gerard (1993) Proc. Natl. Acad. Sci. U.S.A. 90:2812-2816). These viruses were amplified and purified for injection into animals using previously described procedures (Becker, T. C., et al. (1994) Methods Cell Biol. 43:161-189).

### EXAMPLE 2

### Animal Studies

Male Wistar rats (Charles River Laboratories, Wilmington, Mass.) weighing 250-300 g were housed on a 12-hour light-dark cycle and were allowed free access to water and standard laboratory chow (65% carbohydrate, 4% fat, 245 protein; Harlan Tekland laboratory diet 9100). These animals were injected with a single moderate dose of streptozotocin (60 mg/kg; Sigma S-0130) intraperitoneally (i.p.), followed by daily monitoring of blood glucose levels in the ad libitum fed state, using an automated blood glucose analyzer (Hemocue AB, Angelholm Sweden). Only those animals in which blood glucose rose to levels greater than 250 mg/dl within 3 days of STZ injection were studied further. Five days after STZ injection, 0.5×10¹² particles of AdCMV-G_{M}ΔC or AdCMV-βGAL were administered via tail vein injection to rats anesthetized with an i.p. injection of Nembutal (50 mg/kg of body weight; Abbott Laboratories, Chicago, Ill.). As an additional control, some animals received no viral injection. After viral administration, animals were individually caged for daily monitoring of food intake, body weight, and blood glucose levels. Six days after viral administration, animals were sacrificed to allow collection of a large blood sample. Liver and muscle samples were excised, rapidly clamp frozen in liquid nitrogen, and stored at −70° C. for further analysis. In a separate set of STZ-injected and virus treated animals, urine volume and glucose concentration were monitored by four 12 h collections of urine from individual rats, and measurement of urine glucose using a glucose oxidase-based assay (Sigma).

### EXAMPLE 3

### Measurement of RNA Levels in Liver

For analysis of RNA levels, liver samples were ground into powder under liquid nitrogen, and total mRNA was extracted using the TRIzol reagent (Invitrogen; Cat. No. 15596-018). First-strand cDNAs were prepared according to the manufacturer's instructions in a 50 μl total reaction mix using 2 μg of total mRNA, 3.2 μg oligo-p[dT]₁₅, and a cDNA synthesis kit (Roche; Cat. No. 1-483-188). The PCR reactions were carried out using 2 μl of the cDNA synthesis mixture, 0.5 μl of 20 μM stock solutions of the primer pairs for amplification of G_{M}ΔC (5′-primer: 5′-GAAGTACCTGGTCAGAACAGC-3′ (SEQ ID NO: 1); 3′-primer: 5′-CTCTGGCTCAGGTTCCTTCTT-3′ (SEQ ID NO: 2)), glucokinase (5′-primer: 5′-TGGCCACAATGATCTCCTGCT-3′ (SEQ ID NO: 3); 3′-primer: 5′-GGCTTTCGCGCATGCGATTTA-3′ (SEQ ID NO: 4)), or the internal control α-tubulin (5′-primer: 5′-GCGTGAGTGTATCTCCATCCA-3′ (SEQ ID NO: 5); 3′-primer: 5′-GGTAGGTGCCAGTGCGAACTT-3′ (SEQ ID NO: 6)), and a PCR reaction mix containing 2.5 units of Taq polymerase (Roche; Cat. No. 1-418-432), and 2 μl of 10 mM dNTP (Roche; Cat. No. 1-814-362) in a 50 μl total reaction mix. The annealing temperature was 45° C. and PCR reaction mix was collected for analysis after a variable number of cycles (25 cycles for G_{M}ΔC and 32 cycles for glucokinase). The synthesized PCR products were run on a 1% agarose gel, stained with ethidium bromide, and detected by UV light using the VersaDoc imaging system (BioRad; Model 5000). The low DNA mass ladder (Gibco; Cat. No. 10068-013) was used as a size standard, and the PCR products were quantified using the QuantityOne program (BioRad).

### EXAMPLE 4

### Immunoblot Analysis and Glycogen Measurements in Liver Samples

Powdered liver samples were homogenized in lysis buffer (50 mM Tris-HCl, pH 7.5, 5 mM EDTA, 1% Triton X-100, and proteinase inhibitors) using a Polytron Homogenizer (VWR Inc., Model PT10-35), followed by two rounds of freeze-thawing. Cell lysates were centrifuged at 3,000×g for 5 minutes and total protein concentration was measured by the Bradford method (Bradford (1976) Anal. Biochem. 72:248-254). Glycogen was measured by extraction in 10% trichloroacetic acid, precipitation with methanol, and digestion of glycogen to free glucose by incubation with 0.4 mg/ml amyloglucosidase, as previously described (Yang et al. (2000) Mol. Cell. Biol. 20:2706-2717).

For analysis of G_{M}ΔC protein levels, samples were centrifuged at 8,000×g for 2 minutes and 2 mg total proteins were incubated with 10 μg anti-FLAG antibody in 1 ml buffer A (PBS, 2% BSA, 5 mM EDTA, and 100 μM PMSF) at 4° C. for 1 h. Then, 20 μl of Ezview Red anti-FLAG M2 affinity gel (Sigma; Cat. No. F-2426) was added and incubated at 4° C. for 1 h. Samples were centrifuged at 8,000×g and washed three times with buffer. The pellets were mixed with 50 μl SDS-running buffer and boiled for 5 minutes. Samples were resolved on SDS-polyacrylamide gels and transferred to nitrocellulose membranes. The membranes were incubated in blocking buffer (50 mM Tris-HCl, pH 7.5, 150 mM NaCl, 1% BSA) for one hour and treated overnight at 4° C. with rabbit polyclonal sera specific for G_{M} (Tang et al. (1991) J. Biol. Chem. 266:15782-15789; a generous gift of Dr. Anna A. Depaoli Roach, University of Indiana Medical Center) at a dilution of 1:1000. The membranes were washed and subsequently treated with horseradish peroxidase-labeled anti-rabbit IgG secondary antibody at 4° C. for 2 h. The protein-antibody complexes were visualized using an enhanced chemiluminescence (ECL) detection kit (NEN Life Science). Glucokinase protein levels were analyzed as described previously (Yang et al. (2002) J. Biol. Chem. 277:1514-1523), using a rabbit polyclonal anti-glucokinase antibody (Becker et al. (1996) J. Biol. Chem. 271:390-394) at a dilution of 1:5000.

### EXAMPLE 5

### Analysis of Plasma Variables

Plasma insulin, glucagon, and leptin levels were measured by RIA kits (Linco Research, St. Charles, Mo., USA; Cat. No. RI-13K, GL-32K, and RL-83K, respectively). Plasma aspartate-aminotransferase (PGOT), triglycerides, ketones, and lactate were measured using kits (Sigma Diagnostics; procedure No. DG158-UV, 337, 310-UV, 735, respectively) as previously described (Gasa et al. (2002) J. Biol. Chem. 277:1524-1530). Plasma FFAs were measured using the FFA-Half micro test kit (Roche; Cat. No. 1-383-175).

### EXAMPLE 6

### Statistical Analysis

Data are expressed as the mean±SEM. Statistical significance was determined by unpaired Student's t test using the statistics module of Microsoft Excel (Microsoft Excel X for Mac; Microsoft Corp., Redmond, Wash., USA). Statistical significance was assumed at p<0.05.

### EXAMPLE 7

### Expression of G_{M}ΔC in Liver of STZ-Treated Rats

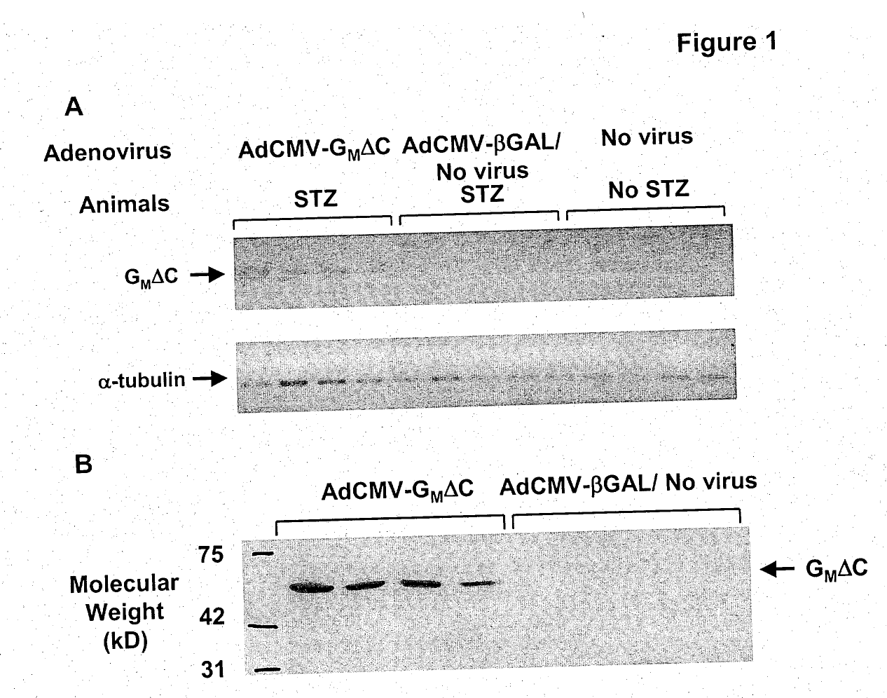

Prior work has shown that expression of G_{M}ΔC in liver of rats with diet-induced obesity and insulin resistance results in reversal of glucose intolerance, in concert with stimulation of liver glycogen storage (Gasa et al. (2002) J. Biol. Chem. 277: 1524-1530). However, these studies did not address the utility of the targeting subunit to correct blood glucose levels in full-blown diabetes. To investigate this point, AdCMV-G_{M}ΔC, or AdCMV-βGAL as a control was injected into Wistar rats that received a single moderate dose of streptozotocin (60 mg/kg). Only animals in which blood glucose exceeded a level of 250 mg/dl within 3 days of STZ injection (ad-lib fed state), and with blood aspartyl aminotransferase (PGOT) activity of less than 200 U/ml after viral injection (indicative of the absence of virus-induced liver damage) were used for further study. As a first step in this analysis, confirmation of the expression of G_{M}ΔC specifically in animals injected with the AdCMV-G_{M}ΔC virus was sought. The cDNA insert contained in this virus includes a FLAG tag to allow specific identification of the virus-encoded protein. As shown in FIG. 1, multiplex PCR analysis of liver RNA reveals an RNA-derived band of expected size only in animals that received the AdCMV-G_{M}ΔC virus, and not in control animals (receiving either AdCMV-βGAL or no virus). Similarly, a protein detectable with an anti-FLAG antibody was immunoprecipitated and immunoblotted only in liver samples from AdCMV-G_{M}ΔC injected animals. It should be noted that it is expected that systemic administration of AdCMV-G_{M}ΔC will result in expression of G_{M}ΔC primarily in the liver, based on prior analysis of a wide array of tissues in rats and mice infused with this and other recombinant adenoviruses (Gasa et al. (2002) J. Biol. Chem. 277: 1524-1530, Herz and Gerard (1993) Proc. Natl. Acad. Sci. U.S.A. 90:2812-2816, O'Doherty et al. (1999) Diabetes 48:2022-2027, Trinh et al. (1998) J. Biol. Chem. 273:31615-31620).

### EXAMPLE 8

### Normalization of Blood Glucose Levels in STZ-Treated Rats by Hepatic Expression of G_{M}ΔC

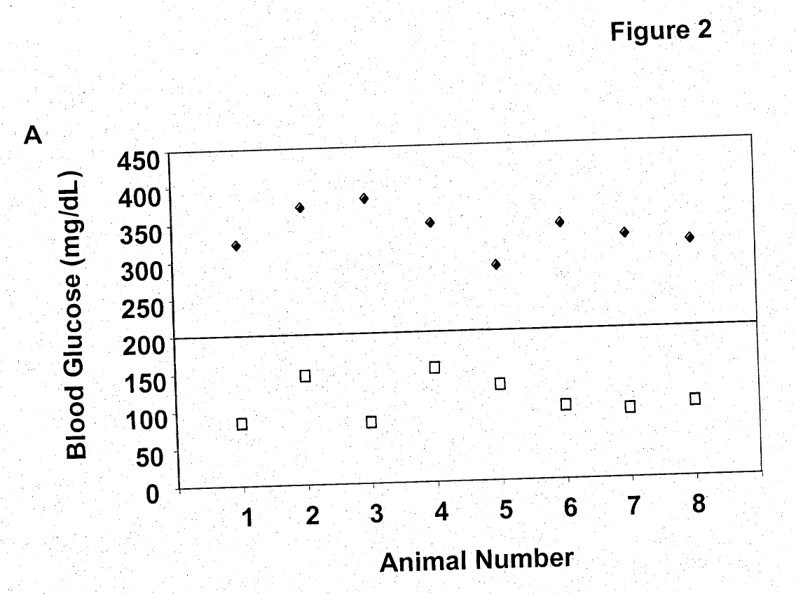

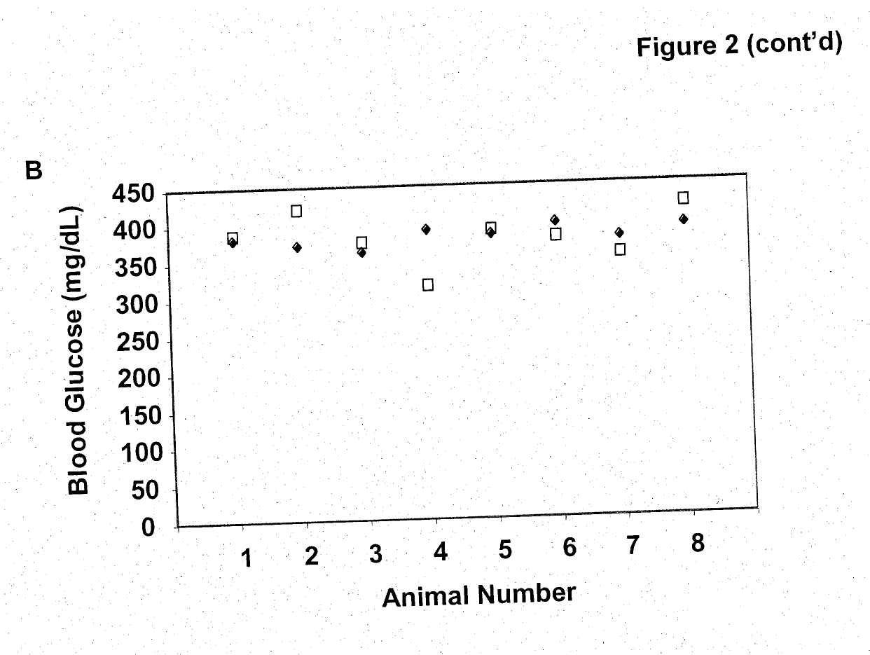

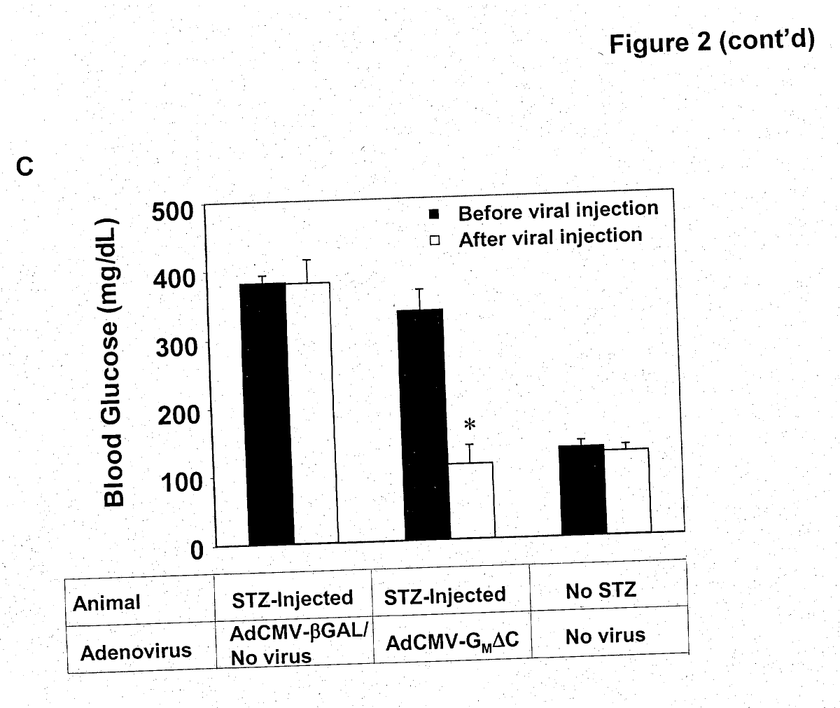

Blood glucose was measured daily for five days after streptozotocin treatment, and then for an additional six days after adenoviral injection. The glucose values for individual STZ-treated animals are shown immediately before and 6 days after injection of AdCMV-G_{M}ΔC (FIG. 2A) or in a control group injected with AdCMV-βGAL or left uninjected (FIG. 2B). All animals that received the AdCMV-G_{M}ΔC virus experienced a decline in blood glucose to the normal range, while this never occurred in AdCMV-βGAL injected or uninjected controls. A summation of this data is provided in FIG. 2C, and shows that control STZ-injected animals had no decline in average glucose values (381±11 versus 379±35 mg/dl prior to and 6 days after viral injection, respectively). In sharp contrast, AdCMV-G_{M}ΔC-injected animals experienced a decline in blood glucose from 335±31 prior to viral injection to 109±28 mg/dl 6 days after injection, the latter value being indistinguishable from the average in normal controls (122±10 mg/dl). Thus, AdCMV-G_{M}ΔC injection completely normalized blood glucose levels in STZ-diabetic rats.

### EXAMPLE 9

### Effect of G_{M}ΔC Expression on Circulating Hormone and Metabolite Levels

Lowering of blood glucose levels by expression of genes that regulate carbohydrate metabolism in liver has the potential to perturb lipid homeostasis, as was evident in a prior study involving adenovirus-mediated expression of glucokinase (O'Doherty et al. (1999) Diabetes 48:2022-2027). Table 1 presents a profile of several key blood hormones and metabolites in the various groups of rats from the current study. With regard to key metabolic regulatory hormones, STZ injection resulted in a 68% decrease in circulating insulin levels relative to uninjected controls, with a similar decrease occurring in STZ-treated animals that received the AdCMV-G_{M}ΔC virus. These decreases in circulating insulin were accompanied by a 40% increase in glucagon levels in both groups. Thus, both groups of STZ-treated rats experienced a fall in insulin:glucagon ratio of approximately 75% (from 0.12 to 0.03), despite the fact that one group was hyperglycemic (AdCMV-βGAL/uninjected controls) and the other normoglycemic (AdCMV-G_{M}ΔC-injected animals). Thus, lowering of blood glucose in the AdCMV-G_{M}ΔC-treated group was achieved by a mechanism independent of changes in insulin:glucagon ratio.

Table 1 also shows that there were no significant changes in circulating triglycerides, free fatty acids, ketones, or lactate in either group of STZ-injected animals (AdCMV-βGAL/uninjected or AdCMV-G_{M}ΔC-injected) relative to control rats that did not receive STZ. Presumably the absence of an increase in circulating lipids or ketones in the STZ-treated groups is attributable to the use of a single moderate dose of the drug that allows some residual insulin production. In sum, lowering of blood glucose by hepatic G_{M}ΔC expression does not perturb other indices of lipid or carbohydrate homeostasis measured in this study.

TABLE 1
Blood metabolite and hormone levels. Animals received
either a single bolus of 60 mg/kg streptozotocin (STZ) or
no streptozotocin injection (No STZ). One group of STZ-
injected rats were treated with AdCMV-βGAL adenovirus
or received no viral treatment (AdCMV-βGAL/No virus).
A separate group of STZ-injected rats received the
AdCMV-G_{M}ΔC virus. Blood samples were taken
six days after viral treatment and used for measurement
of the indicated metabolites and hormones. Data represent
the mean ± SEM for 8 animals in each of the STZ-
treated groups and 6 animals in the No STZ group.
The symbols *** and * indicate significant differences
relative to the No STZ group, with p < 0.001
and 0.05, respectively.

| | STZ | STZ AdCMV-βGAL/ | No STZ |
|---|---|---|---|
| Adenovirus | G_{M}ΔC | No Virus | No Virus |
| Insulin (ng/dL) | 1.64 ± 0.6*** | 1.35 ± 0.35*** | 4.12 ± 0.76 |
| Glucagon (pg/ml) | 47.9 ± 10.3* | 46.4 ± 5.7* | 33.1 ± 2.4 |
| TG (mg/dL) | 61.3 ± 10.2 | 51.8 ± 14.5 | 51.1 ± 14.6 |
| FFA (uM) | 36.6 ± 23.9 | 32.9 ± 9.7 | 45.2 ± 16.8 |
| Ketone (mg/dL) | 1.30 ± 0.30 | 2.00 ± 0.41 | 1.47 ± 0.25 |
| Lactate (mM) | 1.72 ± 0.45 | 2.05 ± 0.48 | 1.13 ± 0.18 |

### EXAMPLE 10

### Normalization of Food Intake by Hepatic Expression of G_{M}ΔC Occurs by a Leptin-Independent Mechanism

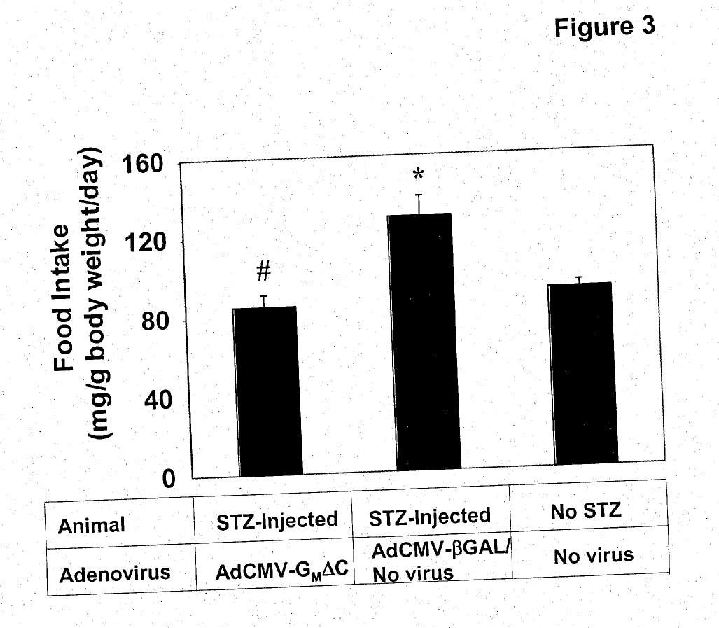

During routine monitoring of food consumption during these studies, a potent effect of G_{M}ΔC expression was observed on this variable. Many groups have previously reported increased food intake in response to STZ injection and type 1 diabetes, and consistent with this, the STZ-injected rats in this study consumed 42% more food on a daily basis than uninjected controls (FIG. 3). Remarkably, injection of AdCMV-G_{M}ΔC into STZ-treated rats caused food consumption to return to normal (FIG. 3).

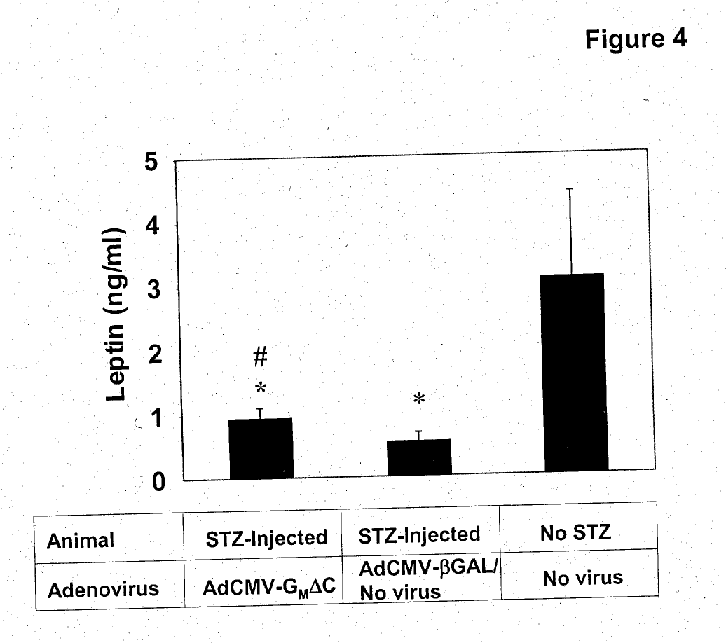

The hormone leptin plays a major role in control of food intake and feeding behavior. Therefore, it was investigated whether the reduction in food intake in G_{M}ΔC expressing animals was secondary to changes in circulating leptin levels. As shown in FIG. 4, STZ injection caused leptin levels to decrease by 82% and 70% in the AdCMV-βGAL-treated/untreated and AdCMV-G_{M}ΔC-treated groups, respectively. On average, leptin levels were slightly but significantly higher in AdCMV-G_{M}ΔC-injected animals than in the control STZ-treated rats (p<0.01). However, it is not believed that this slight elevation in leptin is of any functional significance. This conclusion is based on the analysis of a subset of several AdCMV-G_{M}ΔC (n=4) or control (n=3) STZ-treated-animals with no significant differences in their plasma leptin levels (0.70±0.15 vs 0.74±0.01 ng/ml in G_{M}ΔC expressing versus control animals, respectively). Despite the indistinguishable circulating leptin levels in these subgroups, food intake was still dramatically reduced in the G_{M}ΔC expressing animals relative to controls (86±4.8 vs 126±2.5 mg/g/day, respectively). Furthermore, work from another laboratory shows that STZ treatment does not influence leptin sensitivity in rats (Sindelar et al. (1999) Diabetes 48:1275-1280). These results suggest that hepatic expression of G_{M}ΔC normalizes the hyperphagia of STZ-injected animals via a leptin-independent mechanism.

One possible explanation for the decline in food intake in AdCMV-G_{M}ΔC-treated rats could be that the lowering of blood glucose in these animals prevented spilling of calories in the form of glucose in the urine, thus avoiding the need for compensatory hyperphagia. To test this idea, urine volume and glucose concentration was measured in a separate group of STZ-injected rats with and without G_{M}ΔC expression. Animals treated with STZ and AdCMV-G_{M}ΔC had blood glucose levels of 316±50 mg/dl immediately prior to and 172±67 mg/dl 6 days after viral injection (n=3). Control animals not injected with AdCMV-G_{M}ΔC had blood glucose levels of 320±54 and 317±44 mg/dl at the same time points (n=8). As measured in the 24 h period between the fifth and sixth day after AdCMV-G_{M}ΔC injection, total urine volume and urine glucose dropped from 78 to 27 ml and from 7.6 g glucose/24 h to 0.9 g glucose/24 h, respectively, in response to hepatic G_{M}ΔC expression. For comparison, animals that did not receive an injection of STZ had blood glucose levels of 120 mg/dl, no detectable glucose in their urine, and produced 18 ml of urine/day. The difference in glucose spilling in the untreated versus AdCMV-G_{M}ΔC-treated STZ-injected animals of 6.7 g glucose/24 h (7.6-0.9 g) is equivalent to a loss of 26 kcal/day. This is nearly exactly matched by the reduction in food intake of 25 kcal/day in G_{M}ΔC expressing STZ-injected rats versus controls. Thus, the reversal of caloric spilling in the urine is accounted for by a compensatory reduction in food intake in STZ-treated rats with hepatic expression of G_{M}ΔC.

### EXAMPLE 11

### Expression of G_{M}ΔC in Liver of STZ-Treated Rats Restores Glycogen Levels to Normal

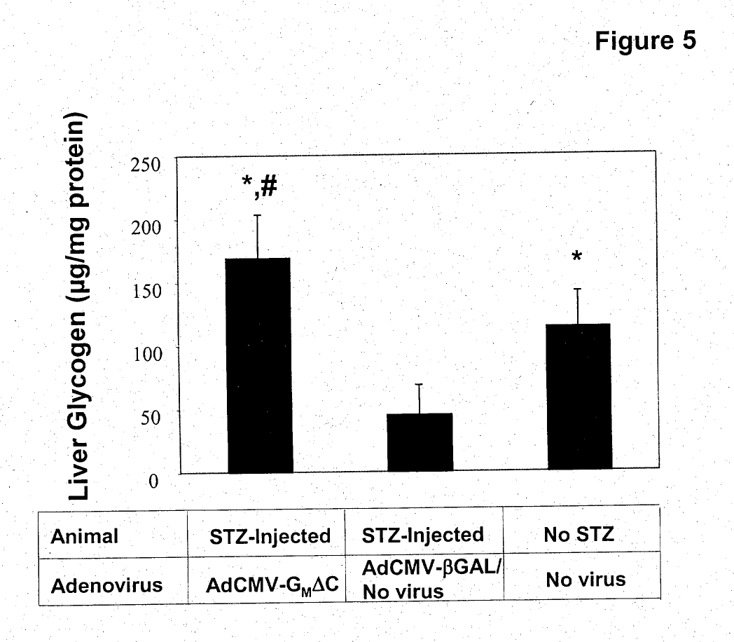

Hepatic glycogen metabolism is impaired in all forms of diabetes (Magnusson et al. (1992) J. Clin. Invest. 90: 1323-1327, Cline et al. (1994) J. Clin. Invest. 94:2369-2376, Velho et al. (1996) J. Clin. Invest. 98:1755-1761). Therefore, the effect of G_{M}ΔC expression on liver glycogen stores in STZ-treated rats was investigated. As shown in FIG. 5, STZ treatment caused a significant 61% decrease in liver glycogen content relative to untreated controls (p<0.001), despite significantly higher glucose levels in the STZ-treated animals (379±35 vs 122±10 mg/dL). In contrast, expression of G_{M}ΔC in liver of STZ-treated rats increased by four-fold relative to STZ-treated controls and by 47% relative to untreated controls (p,0.01). Thus, expression of G_{M}ΔC stimulated liver glycogen deposition in STZ-injected rats, thereby contributing to lowering of blood glucose levels.

### EXAMPLE 12

### G_{M}ΔC Expression in Liver of STZ-Treated Rats Exerts Metabolic Effects Without Affecting Glucokinase Expression

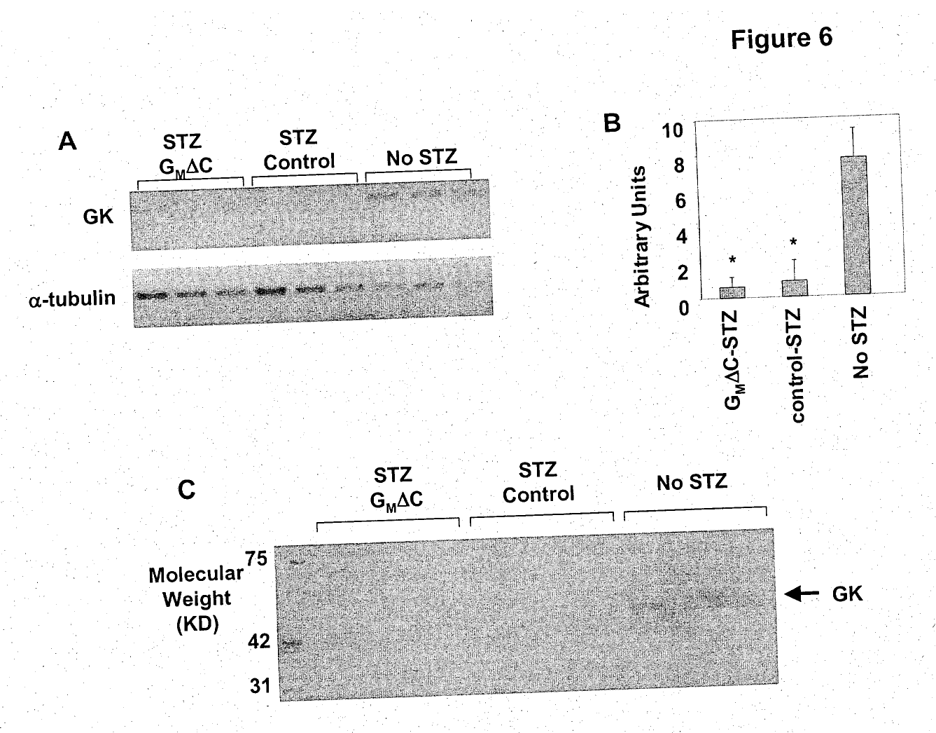

Insulin deficient states are known to be associated with a sharp decline in hepatic glucokinase expression (Iynedjian (1993) Biochem. J. 293:1-13. Printz et al. (1993) Annu. Rev. Nutr. 13:463-496, Matschinsky (1996) Diabetes 45:223-2241), and reduction of glucokinase activity by liver-specific gene knock-out in mice results in impaired hepatic glycogen storage and glucose intolerance (Postic et al. (1999) J. Biol. Chem. 274:305-315). These findings prompted us to investigate glucokinase expression in the model system described in this study. As shown in FIG. 6, STZ injection caused a sharp reduction in glucokinase mRNA and protein levels, as measured by RT-PCR and immunoblot analysis, respectively. Remarkably, glucokinase mRNA and protein levels were also very low in AdCMV-G_{M}ΔC-injected, STZ-treated rats. Glucokinase-catalyzed phosphorylation of glucose is normally perceived as an important regulatory step in hepatic glucose balance.

The foregoing is illustrative of the present invention, and is not to be construed as limiting thereof. The invention is defined by the following claims, with equivalents of the claims to be included therein.

That which is claimed is:

1. A method of reducing caloric intake by a subject comprising administering to the subject a compound that enhances glycogen synthesis but does not substantially impair responsiveness to glycogenolytic signals, wherein the compound is administered in an amount effective to reduce caloric intake in the subject.

2. The method of claim 1, wherein the method comprises administering a compound that enhances hepatic glycogen synthesis but does not substantially impair hepatic responsiveness to glycogenolytic signals.

3. The method of claim 1, wherein the subject has a condition marked by hyperglycemia.

4. The method of claim 1, wherein the subject has diabetes mellitus.

5. The method of claim 1, wherein the subject has hyperphagia.

6. The method of claim 1, wherein the subject is obese.

7. The method of claim 1, wherein the subject has low hepatic glucokinase activity.

8. The method of claim 1, wherein the compound is a polypeptide.

9. The method of claim 1, wherein the compound is an antibody.

10. The method of claim 1, wherein the compound is a nucleic acid molecule.

11. The method of claim 10, wherein the compound is a DNA molecule.

12. The method of claim 11, wherein the compound is a DNA molecule encoding a carboxyl-terminal deleted G_{M} subunit.

13. The method of claim 10, wherein the compound is an RNA molecule.

14. The method of claim 1, wherein the compound binds to a G_{M} subunit.

15. The method of claim 1, wherein the compounds is selected from the group consisting of a substituted n-(indole-2-carbonyl-) amide and a substituted n-(indole-2-carbonyl-) glycinamide.

16. The method of claim 1, wherein the compound is administered to the liver.

17. The method of claim 1, wherein the compound is identified by a process comprising:
contacting a cell with a compound under conditions whereby glycogen synthesis and glycogenolysis can be detected; and
detecting whether the compound enhances glycogen synthesis but does not substantially impair responsiveness to glycogenolytic signals, thereby identifying a compound that can reduce caloric intake.

18. The method of claim 1, wherein the compound is identified by a process comprising:
administering a compound to a subject under conditions whereby glycogen synthesis and glycogenolysis can be detected; and
detecting whether the compound enhances glycogen synthesis but does not substantially impair responsiveness to glycogenolytic signals, thereby identifying a compound that can reduce caloric intake.

19. A method of identifying a compound that can reduce caloric intake, comprising:
contacting a cell with a compound under conditions whereby glycogen synthesis and glycogenolysis can be detected; and
detecting whether the compound enhances glycogen synthesis but does not substantially impair responsiveness to glycogenolytic signals, thereby identifying a compound that can reduce caloric intake.

20. The method of claim 19, wherein the cell is a hepatocyte.

21. The method of claim 19, wherein the compound is a polypeptide.

22. The method of claim 19, wherein the compound is an antibody.

23. The method of claim 19, wherein the compound is a nucleic acid molecule.

24. The method of claim 23, wherein the compound is a DNA molecule.

25. The method of claim 24, wherein the compound is a DNA molecule encoding a carboxyl-terminal deleted G_{M} subunit.

26. The method of claim 23, wherein the compound is an RNA molecule.

27. The method of claim 19, wherein the compound binds to a G_{M} subunit.

28. A method of identifying a compound that can reduce caloric intake, comprising:
administering a compound to a subject under conditions whereby glycogen synthesis and glycogenolysis can be detected; and
detecting whether the compound enhances glycogen synthesis but does not substantially impair responsiveness to glycogenolytic signals, thereby identifying a compound that can reduce caloric intake.

29. The method of claim 28, wherein the subject is a non-human mammal.

30. The method of claim 29, wherein the subject is an animal model for diabetes mellitus.

31. The method of claim 28, wherein the subject is a human.

32. The method of claim 28, wherein the subject has hyperphagia.

33. A method of reducing caloric intake in a subject with diabetes mellitus comprising, administering to the subject an isolated nucleic acid comprising a nucleotide sequence encoding a carboxyl-terminal deleted G_{M} subunit in an amount effective to reduce caloric intake.

34. The method of claim 33, wherein the subject is a non-human mammal.

35. The method of claim 33, wherein the subject is a human.

36. The method of claim 33, wherein the subject has hyperphagia.

37. The method of claim 33, wherein the subject is obese.

38. The method of claim 33, wherein the subject has low hepatic glucokinase activity.

39. The method of claim 33, wherein the nucleic acid further comprises a transcriptional control element functional in hepatocytes and which is operably associated with the nucleotide sequence encoding the carboxyl-terminal deleted G_{M} subunit.

40. The method of claim 33, wherein the nucleic acid is administered to the subject in a delivery vector.

41. A method of reducing hyperglycemia in a subject comprising administering to the subject a compound that enhances glycogen synthesis but does not substantially impair responsiveness to glycogenolytic signals, wherein the compound is administered in an amount effective to reduce hyperglycemia in the subject.

42. The method of claim 41, wherein the method comprises administering a compound that enhances hepatic glycogen synthesis but does not substantially impair hepatic responsiveness to glycogenolytic signals

43. The method of claim 41, wherein the subject has diabetes mellitus.

44. The method of claim 41, wherein the subject is a non-human mammal.

45. The method of claim 44, wherein the subject is an animal model for diabetes mellitus.

46. The method of claim 41, wherein the subject is a human.

47. The method of claim 41, wherein the subject has low hepatic glucokinase activity.

48. The method of claim 41, wherein the compound is a polypeptide.

49. The method of claim 41, wherein the compound is an antibody.

50. The method of any claim 41, wherein the compound is a nucleic acid molecule.

51. The method of claim 50, wherein the compound is a DNA molecule.

52. The method of claim 51, wherein the compound is a DNA molecule encoding a carboxyl-terminal deleted G_{M} subunit.

53. The method of claim 50, wherein the compound is an RNA molecule.

54. The method of claim 41, wherein the compound binds to a G_{M} subunit.

55. The method of claim 41, wherein the compound is administered to the liver.

56. The method of claim 41, wherein the compound is identified by a process comprising:
contacting a cell with a compound under conditions whereby glycogen synthesis and glycogenolysis can be detected; and
detecting whether the compound enhances glycogen synthesis but does not substantially impair responsiveness to glycogenolytic signals, thereby identifying a compound that can reduce hyperglycemia in a subject with diabetes mellitus.

57. The method of claim 41, wherein the compound is identified by a process comprising:
administering a compound to a subject under conditions whereby glycogen synthesis and glycogenolysis can be detected; and
detecting whether the compound enhances glycogen synthesis but does not substantially impair responsiveness to glycogenolytic signals, thereby identifying a compound that can reduce hyperglycemia in a subject with diabetes mellitus.

58. The method of claim 57, wherein the compound is administered to a subject with diabetes mellitus.

59. A method of identifying a compound that can reduce hyperglycemia in a subject with diabetes mellitus, comprising:
contacting a cell with a compound under conditions whereby glycogen synthesis and glycogenolysis can be detected; and
detecting whether the compound enhances glycogen synthesis but does not substantially impair responsiveness to glycogenolytic signals, thereby identifying a compound that can reduce hyperglycemia in a subject with diabetes mellitus.

60. The method of claim 59, wherein the cell is a hepatocyte.

61. The method of claim 59, wherein the compound is a polypeptide.

62. The method of claim 59, wherein the compound is an antibody.

63. The method of claim 59, wherein the compound is a nucleic acid molecule.

64. The method of claim 63, wherein the compound is a DNA molecule.

65. The method of claim 63, wherein the compound is an RNA molecule.

66. The method of claim 59, wherein the compound binds to a G_{M} subunit.

67. A method of identifying a compound that can reduce hyperglycemia in a subject with diabetes mellitus, comprising:
administering a compound to a subject under conditions whereby glycogen synthesis and glycogenolysis can be detected; and
detecting whether the compound enhances glycogen synthesis but does not substantially impair responsiveness to glycogenolytic signals, thereby identifying a compound that can reduce hyperglycemia in a subject with diabetes mellitus.

68. The method of claim 67, wherein the compound is administered to a subject with diabetes mellitus.

69. A method of reducing hyperglycemia in a subject with diabetes mellitus, comprising, administering to the subject an isolated nucleic acid comprising a nucleotide sequence encoding a carboxyl-terminal deleted G_{M} subunit in an amount effective to reduce hyperglycemia.

70. The method of claim 69, wherein the subject is a non-human mammal.

71. The method of claim 69, wherein the subject is an animal model for diabetes mellitus.

72. The method of claim 69, wherein the subject is a human.

73. The method of claim 69, wherein the subject has low hepatic glucokinase activity.

74. The method of claim 69, wherein the nucleic acid further comprises a transcriptional control element functional in hepatocytes and which is operably associated with the nucleotide sequence encoding the carboxyl-terminal deleted G_{M} subunit.

75. The method of claim 69, wherein the nucleic acid is administered to the subject in a delivery vector.
